(19) United States
(12) Patent Application Publication
(10) Pub. No.: US 2025/0314516 A1
(43) Pub. Date: Oct. 9, 2025

(54) SENSOR

(71) Applicant: LG ELECTRONICS INC., Seoul (KR)

(72) Inventors: Mangeun KIM, Seoul (KR); Sunghyun YI, Seoul (KR); Hyunjin PARK, Seoul (KR)

(73) Assignee: LG ELECTRONICS INC., Seoul (KR)

(21) Appl. No.: 19/098,732

(22) Filed: Apr. 2, 2025

(30) Foreign Application Priority Data
Apr. 3, 2024 (KR) 10-2024-0045162
Jul. 24, 2024 (KR) 10-2024-0097754
Sep. 10, 2024 (KR) 10-2024-0123069
Apr. 1, 2025 (WO) PCT/KR2025/004301

(51) Int. Cl. G01F 23/263; G01N 27/22; G01N 33/28

(52) U.S. Cl. CPC G01F 23/263; G01N 27/226; G01N 33/2888

(57) ABSTRACT

The present disclosure relates to a sensor. The sensor according an aspect of the present disclosure may include: a cap; electrodes facing the cap and spaced apart from each other; and pins penetrating the cap and electrically connected to the electrodes, wherein the electrodes may include: an upper electrode in a shape of a vertical plate; a first ground electrode facing one surface of the upper electrode; a lower electrode located below the upper electrode; and a shield electrode disposed between the upper electrode and the lower electrode, wherein the shield electrode may cover a lower edge of the upper electrode and a lower edge of the first ground electrode.

### CROSS-REFERENCE TO RELATED APPLICATIONS

Pursuant to 35 U.S.C. § 119, this application claims the benefit of earlier filing date and right of priority to Korean Patent Application Nos. 10-2024-0045162, filed on Apr. 3, 2024, 10-2024-0097754, filed on Jul. 24, 2024, and 10-2024-0123069, filed on Sep. 10, 2024, and International Application No. PCT/KR2025/004301, filed on Apr. 1, 2025, the contents of which are all hereby incorporated by reference herein in their entireties.

### BACKGROUND OF THE INVENTION

### Field of the Invention

This disclosure relates to a sensor, and more particularly, to a sensor for calculating the amount of oil existing inside a compressor.

### Description of the Related Art

A compressor is installed in a home appliance such as a refrigerator and an air conditioner or in a vehicle to compress refrigerant. The compressor is connected to a condenser and an evaporator, and can compress a refrigerant evaporated from the evaporator and supply it to the condenser.

In order to protect the compressor from mechanical friction, lubrication or cooling is performed through oil, and the compressor must always have a certain level of oil or more. The oil inside the compressor circulates through a refrigerant cycle together with the refrigerant discharged from the compressor. At this time, if the oil accumulates in the condenser, evaporator, and pipe of the refrigerant cycle, it will cause a decrease in the system's capacity, and if the oil recovery is not smooth, the amount of oil inside the compressor will be insufficient, which may cause damage to the compressor. In order to prevent such damage to the compressor, the system performs an oil recovery operation to recover the oil accumulated in the condenser, evaporator, and pipe to the compressor.

In the conventional case, a separate oil level sensor is arranged in the compressor, and oil recovery operation is performed according to the amount of oil detected by the oil level sensor. If an oil level sensor is used, unnecessary oil recovery operation can be reduced, thereby increasing energy efficiency and reliability of compressor performance.

However, according to a conventional method, the oil level sensor is arranged at a certain location inside the compressor, so that it is only possible to check whether the amount of oil existing inside the compressor corresponds to the certain location where the oil level sensor is installed. Thus, there is a problem that a plurality of oil level sensors must be installed at various locations inside the compressor in order to accurately detect the amount of oil existing inside the compressor.

### SUMMARY OF THE INVENTION

The present disclosure aims to solve the aforementioned problems and other problems.

The disclosure may further provide a sensor capable of accurately detecting the oil concentration inside a compressor.

The disclosure may further provide a sensor capable of accurately detecting the height of the oil surface based on the oil concentration inside a compressor.

The disclosure may further provide a sensor capable of accurately detecting the amount of oil existing inside a compressor.

The disclosure may further provide a sensor optimized to detect the oil concentration, the height of the oil surface and the amount of oil using a plurality of electrodes.

The disclosure may further provide various examples of sensing modules of a sensor.

In accordance with another aspect of the present disclosure, a sensor may include: a cap; electrodes facing the cap and spaced apart from each other; and pins penetrating the cap and electrically connected to the electrodes, wherein the electrodes may include: an upper electrode in a shape of a vertical plate; a first ground electrode facing one surface of the upper electrode; a lower electrode located below the upper electrode; and a shield electrode disposed between the upper electrode and the lower electrode, wherein the shield electrode may cover a lower edge of the upper electrode and a lower edge of the first ground electrode.

### BRIEF DESCRIPTION OF THE DRAWINGS

The above and other objects, features and advantages of the present disclosure will be more apparent from the following detailed description in conjunction with the accompanying drawings, in which:

FIG. 1A and FIG. 1B are diagrams illustrating a configuration of a system, according to an embodiment of the present disclosure;

FIG. 2 is a block diagram of a system, according to an embodiment of the present disclosure;

FIG. 3 is a block diagram of a sensor, according to an embodiment of the present disclosure;

FIG. 4 is a diagram illustrating a configuration of an electrode assembly, according to an embodiment of the present disclosure;

FIG. 5 and FIG. 6 are diagrams for explaining a sensor, according to an embodiment of the present disclosure;

FIG. 7 is a diagram illustrating a configuration of an electrode assembly, according to another embodiment of the present disclosure;

FIG. 8 and FIG. 9 are diagrams for explaining a sensor, according to another embodiment of the present disclosure;

FIG. 10 to FIG. 13 are diagrams illustrating a configuration of an electrode assembly, according to embodiments of the present disclosure;

FIG. 14 to FIG. 21 are diagrams illustrating a configuration of an electrode assembly, according to embodiments of the present disclosure; and

FIG. 22 to FIG. 28 are diagrams illustrating a sensing module of a sensor, according to an embodiment of the present disclosure.

### DETAILED DESCRIPTION

Hereinafter, the present disclosure will be described in detail with reference to the drawings. In the drawings, in order to clearly and concisely describe the present disclosure, parts that are not related to the description are omitted, and the same drawing reference numerals are used for identical or extremely similar parts throughout the specification.

The suffixes “module” and “part” used for components in the following description are given simply for the convenience of writing this specification, and do not in themselves impart any particularly important meaning or role. Therefore, the above “module” and “part” may be used interchangeably.

In the present application, it should be understood that the terms “comprises, includes,” “has,” etc. specify the presence of features, numbers, steps, operations, elements, components, or combinations thereof described in the specification, but do not preclude the presence or addition of one or more other features, numbers, steps, operations, elements, components, or combinations thereof.

In addition, in this specification, terms such as first, second, etc. may be used to describe various elements, but these elements are not limited by these terms. These terms are used only to distinguish one element from another.

Hereinafter, direction is defined based on a rectangular coordinate system. In the rectangular coordinate system, the x-axis direction may be defined as a left-right direction. At this time, based on the origin, the direction toward +x may mean a right direction, and the direction toward −x may mean a left direction. In addition, the y-axis direction may be defined as a front-rear direction. At this time, based on the origin, the direction toward +y may mean a forward direction, and the direction toward −y may mean a rearward direction. In addition, the z-axis direction may be defined as an up-down direction. At this time, based on the origin, the direction toward +z may mean an upward direction, and the direction toward −z may mean a downward direction.

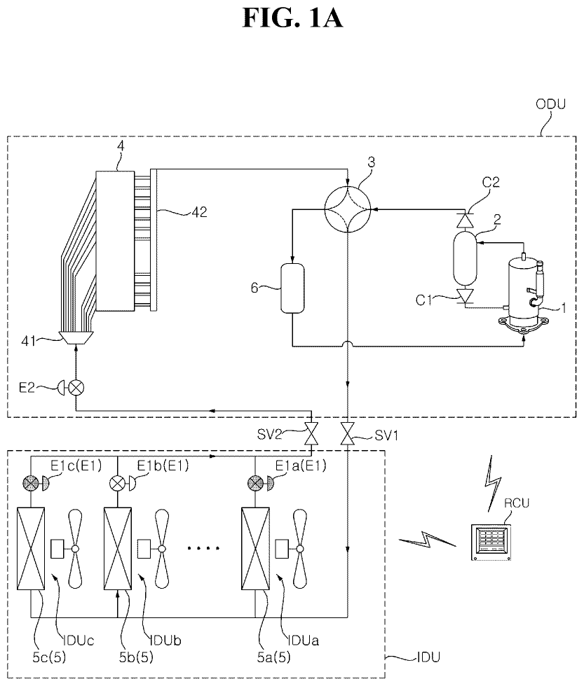

FIG. 1A and FIG. 1B are drawings illustrating a system, according to various embodiments of the present disclosure.

Referring to FIG. 1A and FIG. 1B, the system may include a compressor 1 that compresses a refrigerant. The refrigerant compressed in the compressor 1 may circulate a refrigerant cycle. In this disclosure, it is described that the system is an air conditioner that provides heat-exchanged air to a room based on a refrigerant cycle.

The system may have an outdoor unit (ODU) and an indoor unit (IDU) that are connected to each other by a refrigerant pipe. The system may further have a remote control unit (RCU). The outdoor unit (ODU), the indoor unit (IDU), and/or the remote control unit (RCU) may transmit and receive signals to and from each other.

The outdoor unit (ODU) may have a compressor 1, an oil separator 2, a switching valve 3, an outdoor heat exchanger 4, an outdoor expansion valve E2, and/or an accumulator 6. The indoor unit (IDU) may have an indoor heat exchanger 5 and an indoor expansion valve E1.

The compressor 1 may compress refrigerant flowed in from the accumulator 6 by high temperature and high pressure. For example, the compressor 1 may be an inverter compressor that can control the amount of refrigerant and the discharge pressure of the refrigerant by adjusting an operating frequency. For example, the compressor 1 may be an oil compressor that uses oil as a lubricant.

The oil separator 2 may recover oil from the refrigerant discharged from the compressor 1 and provide it back to the compressor 1. At this time, a first check valve C1 may be installed in a pipe through which the oil separated from the oil separator 2 flows, so as to limit the flow direction of the oil to a direction from the oil separator 2 to the compressor 1.

The switching valve 3 may selectively guide the refrigerant flowed in from the oil separator 2 to the outdoor heat exchanger 4 or the indoor heat exchanger 5. For example, the switching valve 3 may be a four-way valve. At this time, the second check valve C2 may limit the flow direction of the refrigerant to a direction from the oil separator 2 to the switching valve 3.

The outdoor heat exchanger 4 may exchange heat between the refrigerant and the outdoor air. The direction of heat transmission between the refrigerant and the outdoor air in the outdoor heat exchanger 4 may vary depending on the operating mode of the system, i.e., whether it is a heating operation or a cooling operation. An outdoor fan (not shown) is installed in one side of the outdoor heat exchanger 4 and may control the amount of air provided to the outdoor heat exchanger 4.

The indoor heat exchanger 5 may exchange heat between the refrigerant and the indoor air. The direction of heat transmission between the refrigerant and the indoor air in the indoor heat exchanger 5 may vary depending on the operating mode of the system, i.e., whether it is a heating operation or a cooling operation. An indoor fan (not shown) may be installed in one side of the indoor heat exchanger 5 and may control the amount of air provided to the indoor heat exchanger 5.

For example, the indoor heat exchanger 5 may include a plurality of indoor heat exchangers 5a, 5b, 5c. In this case, the indoor unit (IDU) may include a first indoor unit (IDUa) having a first indoor heat exchanger 5a, a first indoor fan, and a first indoor expansion valve E1a, a second indoor unit (IDUb) having a second indoor heat exchanger 5b, a second indoor fan, and a second indoor expansion valve E1b, and a third indoor unit (IDUc) having a third indoor heat exchanger 5c, a third indoor fan, and a third indoor expansion valve E1c. Meanwhile, in response to a required load for indoor cooling or heating, some of a plurality indoor heat exchangers 5a, 5b, 5c may be operated, and the rest may not be operated.

The expansion valve E1, E2 may be installed between the outdoor heat exchanger 4 and the indoor heat exchanger 5, and may expand the refrigerant that has passed through the outdoor heat exchanger 4 or the indoor heat exchanger 5. In addition, the expansion valves E1, E2 may include an outdoor expansion valve E2 adjacent to the outdoor heat exchanger 4 and an indoor expansion valve E1 adjacent to the indoor heat exchanger 5. In this case, the outdoor expansion valve E2 may be used to expand the refrigerant that has passed through the indoor heat exchanger 5, and the indoor expansion valve E1 may be used to expand the refrigerant that has passed through the outdoor heat exchanger 4. For example, the expansion valve E1, E2 may be Electronic Expansion Valve (EEV) capable of controlling the opening degree of the flow path of the refrigerant pipe in which the expansion valve E1, E2 is installed.

For example, the indoor expansion valve E1 may include a first indoor expansion valve E1a that expands the refrigerant provided to the first indoor heat exchanger 5a, a second indoor expansion valve E1b that expands the refrigerant provided to the second indoor heat exchanger 5b, and a third indoor expansion valve E1c that expands the refrigerant provided to the third indoor heat exchanger 5c.

A plurality of sensors (not shown) may measure the temperature and/or pressure of the refrigerant flowing through the refrigerant pipe. A controller (not shown) may be electrically connected to each component of the system, and may control the operation of each component of the system.

Referring to FIG. 1A, when a heating operation signal is input to the system, the controller may perform heating operation of the system. For example, the heating operation signal may be a signal arbitrarily input by a user.

For another example, the heating operation signal may be a signal provided by a thermostat installed in an indoor space to a controller, when the indoor temperature detected by the indoor temperature sensor is lower than a desired temperature set by a user by a certain level or higher.

Specifically, the low-temperature, low-pressure refrigerant flowing from the accumulator 6 to the compressor 1 may be compressed by high temperature, high pressure in the compressor 1 and discharged to the oil separator 2. Then, the refrigerant from which oil is separated in the oil separator 2 may flow into the second indoor heat exchanger 5b via the switching valve 3 and the first service valve SV1. At this time, the second indoor expansion valve E1b may completely open the refrigerant flow path that passes through the second indoor heat exchanger 5b and leads to the outdoor heat exchanger 4. In addition, the first indoor expansion valve E1a and the third indoor expansion valve E1c can close the refrigerant flow path that passes through the first indoor heat exchanger 5a and the third indoor heat exchanger 5c and leads to the outdoor heat exchanger 4. In addition, when a required heating load increases, the first indoor expansion valve E1a and/or the third indoor expansion valve E1c may also be opened.

As heat energy is transmitted from the refrigerant to the indoor air in the second indoor heat exchanger 5b, the refrigerant may be condensed. At this time, the second indoor heat exchanger 5b may serve as a condenser. In addition, as the heat exchange occurs between the refrigerant and the indoor air, the indoor space may be heated. The refrigerant condensed while passing through the second indoor heat exchanger 5b may pass through the outdoor expansion valve E2, via the second indoor expansion valve E1b and a second service valve SV2. The refrigerant expanded while passing through the outdoor expansion valve E2 may be distributed to multiple points of the outdoor heat exchanger 4 via a distributor 41.

As the heat energy of the outdoor air is transmitted to the refrigerant in the outdoor heat exchanger 4, the refrigerant may be evaporated. At this time, the outdoor heat exchanger 4 may serve as an evaporator. The refrigerant evaporated while passing through the outdoor heat exchanger 4 may be flowed into the compressor 1 via the header 42, the switching valve 3, and the accumulator 6 sequentially. Thus, a refrigerant cycle for the heating operation of the aforementioned system can be completed.

Referring to FIG. 1B, when a cooling operation signal is input to the system, the controller may perform the cooling operation of the system. For example, the cooling operation signal may be a signal arbitrarily input by a user.

For another example, the cooling operation signal may be a signal provided by a thermostat installed in the indoor space to the controller, when the indoor temperature detected by the indoor temperature sensor is higher than a desired temperature set by a user by a certain level or higher.

Specifically, the low-temperature, low-pressure refrigerant flowing from the accumulator 6 to the compressor 1 may be compressed by a high temperature, high pressure in the compressor 1 and discharged to the oil separator 2. Then, the refrigerant from which oil is separated in the oil separator 2 may be flowed into the outdoor heat exchanger 4 via the switching valve 3 and the header 42.

As heat energy is transmitted from the refrigerant to the outdoor air in the outdoor heat exchanger 4, the refrigerant may be condensed. At this time, the outdoor heat exchanger 4 may serve as a condenser.

The refrigerant condensed while passing through the outdoor heat exchanger 4 may flow into the second indoor expansion valve E1b via the distributor 41, the outdoor expansion valve E2, and the second service valve SV2 sequentially. At this time, the outdoor expansion valve E2 may completely open the flow path. Then, the refrigerant expanded while passing through the second indoor expansion valve E1b may flow into the second indoor heat exchanger 5b. In addition, when the required cooling load increases, the first indoor expansion valve E1a and/or the third indoor expansion valve E1c may also be opened by a certain degree.

As the heat energy of the indoor air is transmitted to the refrigerant in the second indoor heat exchanger 5b, the refrigerant may evaporate. At this time, the second indoor heat exchanger 5b may serve as an evaporator. Then, the indoor space may be cooled by the heat exchange between the refrigerant and the indoor air. The refrigerant that has evaporated while passing through the second indoor heat exchanger 5b may be flowed into the compressor 1 via the first service valve SV1, the switching valve 3, and the accumulator 6 sequentially. Thus, a refrigerant cycle for cooling operation of the aforementioned system may be completed.

Meanwhile, the system may perform an oil recovery operation. For example, when the system performs an oil recovery operation, the system may control the switching valve 3 so that the refrigerant discharged from the compressor 1 flows to the outdoor heat exchanger 4. At this time, the refrigerant may be condensed by heat exchange between the refrigerant and the outdoor air that occurs in the outdoor heat exchanger 4. The refrigerant condensed in the outdoor heat exchanger 4 may be flowed into the indoor unit (IDU). The refrigerant may be evaporated by heat exchange between the refrigerant flowed into the indoor unit (IDU) and the indoor air.

According to an embodiment, when performing an oil recovery operation, the system may increase the operating frequency of the compressor 1 to a certain level or more. For example, when performing an oil recovery operation, the system may set the operating frequency of the compressor 1 to the maximum value. When performing an oil recovery operation, the system may stop the operation of an indoor fan. When performing an oil recovery operation, the system may control the operation of the outdoor fan, based on the pressure of the outdoor unit-side pipe. For example, if the pressure of the outdoor unit-side pipe exceeds a certain standard, the outdoor fan may be operated, and if it is below the certain standard, the operation of the outdoor fan may be stopped.

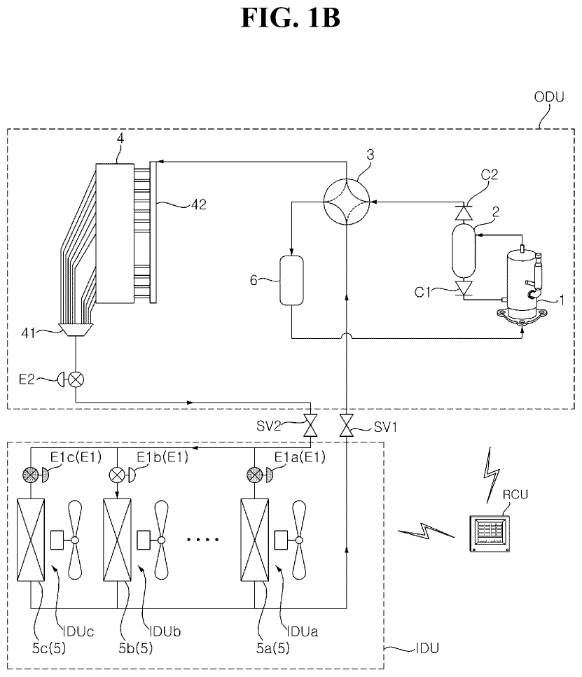

FIG. 2 is a block diagram of a system, according to an embodiment of the present disclosure.

Referring to FIG. 2, the system may include a communication unit 210, a sensor unit 220, a memory 230, a fan driving unit 240 that drives a fan 241, a compressor driving unit 250 that drives a compressor 251 (compressor 1 of FIG. 1A), and/or a controller 260.

The communication unit 210 may include at least one communication module. For example, the communication unit 210 may be provided in each of the outdoor unit (ODU) and the indoor unit (IDU), and the outdoor unit (ODU) and the indoor unit (IDU) may transmit and receive data to and from each other. For example, the communication unit 210 may be provided in the remote control unit (RCU).

The communication method of the outdoor unit (ODU), the indoor unit (IDU), and/or the remote control unit (RCU) may be, for example, a communication method using a power line, a serial communication method (e.g., RS-485 communication), a wired communication method through refrigerant piping, or a wireless communication method such as Wi-fi, Bluetooth, Beacon, and Zigbee.

The communication unit 210 may transmit and receive data to and from an external device. For example, the communication unit 210 may access a server connected to an external network to transmit and receive data.

The sensor unit 220 may have at least one sensor, and may transmit data on a detection value detected through the sensor to the controller 260.

The sensor unit 220 may have a heat exchanger temperature sensor (not shown). For example, the heat exchanger temperature sensor may be arranged inside the indoor heat exchanger 5, and may detect the temperature of the indoor heat exchanger 5.

The sensor unit 220 may have a pipe temperature sensor (not shown). The pipe temperature sensor may detect the temperature of the refrigerant flowing through each pipe of the system. For example, the pipe temperature sensor may be arranged in the inlet-side pipe of the indoor unit (IDU) and/or the outlet-side pipe of the indoor unit (IDU), and may detect the temperature of the refrigerant flowing through the pipe. For example, the pipe temperature sensor may be arranged in a pipe connected to the compressor 251 to detect the temperature (hereinafter, suction temperature) of the refrigerant flowing into the compressor 251 and/or the temperature (hereinafter, discharge temperature) of the refrigerant discharged from the compressor 251.

The sensor unit 210 may have a pressure sensor (not shown). The pressure sensor (not shown) may detect the pressure of the gaseous refrigerant flowing through each pipe of the system. For example, the pressure sensor may be arranged in a pipe connected to the compressor 251 to detect the pressure (hereinafter, suction pressure) of the refrigerant flowing into the compressor 251 and/or the pressure (hereinafter, discharge pressure) of the refrigerant discharged from the compressor 251.

The sensor unit 220 may have an indoor temperature sensor (not shown) that detects the indoor temperature and/or an outdoor temperature sensor (not shown) that detects the outdoor temperature.

The sensor unit 220 may have an indoor humidity sensor (not shown) that detects indoor humidity and/or an outdoor humidity sensor (not shown) that detects outdoor humidity.

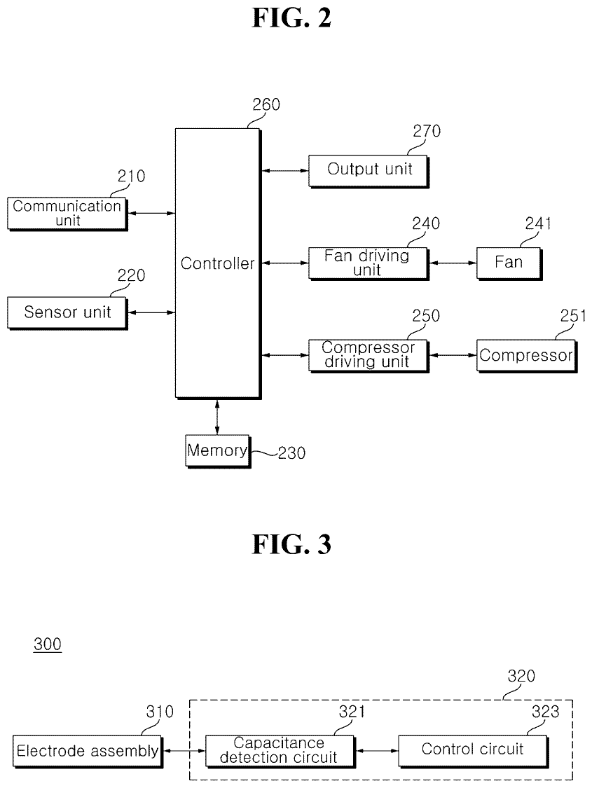

The sensor unit 220 may include a sensor 300 (see FIG. 3) (hereinafter, oil sensor) that outputs a signal corresponding to oil stored inside the compressor 251. The oil sensor 300 may detect the height of the fluid surface inside the compressor 251. The oil sensor 300 may detect the amount of fluid stored inside the compressor 251. The oil sensor 300 may detect the concentration of oil with respect to the fluid. The fluid stored inside the compressor 251 may at least include oil. The fluid stored inside the compressor 251 may further include a refrigerant.

The memory 230 may store data related to the operation of each component provided in the system.

The memory 230 may store programs for processing and controlling each signal within the controller 260, and may store processed data and data to be processed. For example, the memory 230 may store application programs designed for the purpose of performing various tasks that can be processed by the controller 260, and may selectively provide some of the stored application programs when requested by the controller 260.

The memory 230 may include, for example, at least one of volatile memory (e.g., DRAM, SRAM, SDRAM, etc.) and nonvolatile memory (e.g., flash memory, hard disk drive (HDD), solid-state drive (SSD), etc.).

The fan driving unit 240 may drive the fan 241 provided in the system. For example, the fan 241 may include an outdoor fan and/or an indoor fan.

The fan driving unit 240 may include a rectifier (not shown) that rectifies AC power into DC power and outputs it, a DC-link capacitor (not shown) that stores a pulsating voltage from the rectifier, an inverter (not shown) that has a plurality of switching elements and converts and outputs smoothed DC power into three-phase AC power of a certain frequency, and/or at least one motor that drives the fan 241 according to the three-phase AC power output from the inverter.

Meanwhile, the fan driving unit 240 may be provided with separate configurations for driving the outdoor fan and the indoor fan, respectively. For example, the system may include a first fan driving unit for driving the outdoor fan and a second fan driving unit for driving the indoor fan.

The compressor driving unit 250 may drive the compressor 251. The compressor driving unit 250 may include a rectifier (not shown) that rectifies AC power into DC power and outputs it, a DC-link capacitor (not shown) that stores a pulsating voltage from the rectifier, an inverter (not shown) that has a plurality of switching elements, and converts and outputs smoothed DC power into three-phase AC power of a certain frequency, and/or a compressor motor 102b that drives the compressor 251 according to the three-phase AC power output from the inverter.

The controller 260 may control the overall operation of the system. The controller 260 may be connected to each component provided in the system, and may control the overall operation of each component by transmitting and/or receiving signals to/and from each component.

The controller 260 may control the operation of the fan driving unit 240 and change the rotation speed of the fan 241. For example, the fan driving unit 240 may change the rotation speed of the outdoor fan by changing the frequency of the three-phase AC power output to the outdoor fan motor, according to the control of the controller 260. For example, the fan driving unit 240 may change the rotation speed of the indoor fan by changing the frequency of the three-phase AC power output to the indoor fan motor, according to the control of the controller 260.

The controller 260 may control the operation of the compressor driving unit 250 to change the operating frequency of the compressor 251. For example, the compressor driving unit 250 may change the operating frequency of the compressor 251, by changing the frequency of the three-phase AC power output to the compressor motor 102b, according to the control of the controller 260.

The controller 260 may be equipped not only in the outdoor unit (ODU), but also in the indoor unit (IDU), the outdoor unit (ODU), and/or the remote control unit (RCU).

The controller 260 may include at least one processor, and may control the overall operation of the system by using the processor included therein. Here, the processor may be a general processor such as a central processing unit (CPU). Obviously, the processor may be a dedicated device such as an ASIC or another hardware-based processor.

The controller 260 may obtain data related to each component provided in the system. At this time, the controller 260 may obtain data related to each component provided in the system at regular time intervals, according to a certain cycle, in consideration of a computational load.

The controller 260 may perform various computations based on the acquired data, and may control the overall operation of each component provided in the system according to a computational result.

Data related to each component provided in the system may include, for example, the operating frequency of the compressor 251, the suction temperature of the compressor 251, the discharge temperature, the suction pressure, the discharge pressure, the concentration of oil stored inside the compressor 251, the amount of oil, the inlet-side pipe temperature of the indoor unit (IDU), the outlet-side pipe temperature of the indoor unit (IDU), the indoor temperature, the outdoor temperature, the opening amount of the electronic expansion valve (EEV), etc.

Meanwhile, the system may further include an input device (not shown) capable of receiving an user input. For example, when the system receives a user input through an input device (e.g., a touch panel, a key, etc.,) the system may perform an operation corresponding to the received user input.

The system may further include an output unit 270 that outputs a message related to the operating state of the system. For example, the output unit 270 may include a display device such as a display and a light emitting diode LED and/or an audio device such as a speaker and a buzzer.

FIG. 3 is a block diagram of a sensor, according to an embodiment of the present disclosure.

Referring to FIG. 3, the oil sensor 300 may include an electrode assembly 310 and/or a processing circuit 320.

The electrode assembly 310 may include a plurality of electrodes. Some of the plurality of electrodes included in the electrode assembly 310 may be formed to extend vertically. Others of the plurality of electrodes included in the electrode assembly 310 may be formed to extend horizontally.

The electrode assembly 310 may be disposed inside the compressor 1. The electrode assembly 310 may be exposed to a fluid stored inside the compressor 1. At least some of the plurality of electrodes included in the electrode assembly 310 may be arranged to be immersed in the fluid. The fluid stored inside the compressor 1 may at least include oil. The fluid stored inside the compressor 1 may further include a refrigerant.

A voltage may be applied to the electrode assembly 310. A sensing voltage may be applied to some of the plurality of electrodes included in the electrode assembly 310. The sensing voltage may be an AC voltage whose voltage value changes periodically. The sensing voltage may have a certain frequency. A ground voltage may be applied to others of the plurality of electrodes included in the electrode assembly 310.

The processing circuit 320 may be electrically connected to the electrode assembly 310. The processing circuit 320 may apply a voltage to the electrode assembly 310.

The processing circuit 320 may detect capacitance, based on the voltage applied to the electrode assembly 310.

For example, the processing circuit 320 may detect, by using a mutual-capacitance method, capacitance corresponding to a first electrode to which a sensing voltage is applied and a second electrode to which a ground voltage is applied, among a plurality of electrodes included in the electrode assembly 310. The capacitance corresponding to two electrodes among the plurality of electrodes may be detected based on Equation 1.

$$C = {ε}_{0} {ε}_{r} \frac{A}{d} \qquad [ Equation 1 ]$$

Here, C may mean capacitance, ε0 may mean permittivity corresponding to vacuum, εr may mean relative permittivity corresponding to a material between two electrodes, d may mean a distance between two electrodes, and A may mean an area of an electrode in contact with a material.

For example, the processing circuit 320 may detect the capacitance corresponding to a single electrode to which the sensing voltage is applied among the plurality of electrodes included in the electrode assembly 310. At this time, the processing circuit 320 may detect the capacitance corresponding to a single electrode to which the sensing voltage is applied through a self-capacitance method.

The processing circuit 320 may calculate the concentration of oil contained in the fluid stored inside the compressor 1, based on the capacitance for the electrode assembly 310. The processing circuit 320 may calculate the height of the fluid surface inside the compressor 1, based on the capacitance for the electrode assembly 310. The processing circuit 320 may calculate the amount of fluid stored inside the compressor 1, based on the capacitance for the electrode assembly 310.

The processing circuit 320 may include a capacitance detection circuit 321 that detects the capacitance for the electrode assembly 310. The processing circuit 320 may include a control circuit 323 that calculates the oil concentration, the height of the fluid surface and/or the amount of fluid. The capacitance detection circuit 321 and the control circuit 323 may be implemented as one configuration or may be implemented as separate configurations. Meanwhile, the capacitance detection circuit 321 and/or the control circuit 323 may be included in the controller 260 of the system.

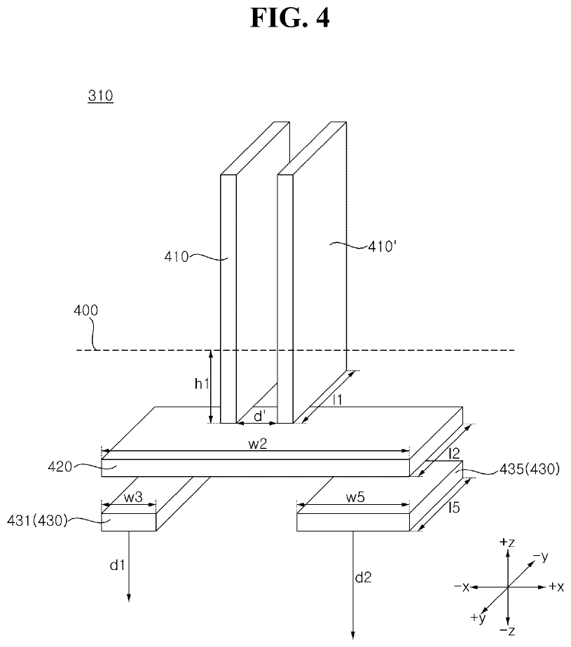

FIG. 4 is a drawing illustrating a configuration of an electrode assembly, according to an embodiment of the present disclosure.

Referring to FIG. 4, the electrode assembly 310 may include a plurality of upper electrodes 410, 410′. The plurality of upper electrodes 410, 410′ may be referred to as an upper electrode assembly 410, 410′.

The plurality of upper electrodes 410, 410′ may be formed to extend vertically. The plurality of upper electrodes 410, 410′ may be formed to have a first length 11 along the front-rear direction.

The plurality of upper electrodes 410, 410′ may be arranged to face each other. One surface of a first upper electrode 410 and one surface of a second upper electrode 410′ may face each other. The plurality of upper electrodes 410, 410′ may be arranged in parallel.

The plurality of upper electrodes 410, 410′ may be arranged to be spaced apart from each other by a certain distance d′. One surface of the first upper electrode 410 and one surface of the second upper electrode 410′ that face each other may be spaced apart by a certain distance d′.

At least some of the plurality of upper electrodes 410, 410′ may come into contact with the fluid stored inside the compressor 1. A sensing voltage may be applied to the first upper electrode 410. A ground voltage may be applied to the second upper electrode 410′. The first upper electrode 410 may be referred to as an upper electrode 410 or a first vertical electrode 410. The second upper electrode 410′ may be referred to as a ground electrode 410′ or a second vertical electrode 410′.

Since the distance d′ between the plurality of upper electrodes 410, 410′ and the length 11 of the plurality of upper electrodes 410, 410′ are constant, the capacitance corresponding to the plurality of upper electrodes 410, 410′ may correspond to the height h1 (hereinafter, fluid level height) from the bottom of the plurality of upper electrodes 410, 410′ to the surface 400 of the fluid. That is, the capacitance corresponding to the plurality of upper electrodes 410, 410′ may correspond to the amount of fluid stored inside the compressor 251.

Meanwhile, the capacitance corresponding to the plurality of upper electrodes 410, 410′ may correspond to the concentration of oil contained in the fluid located between the plurality of upper electrodes 410, 410′.

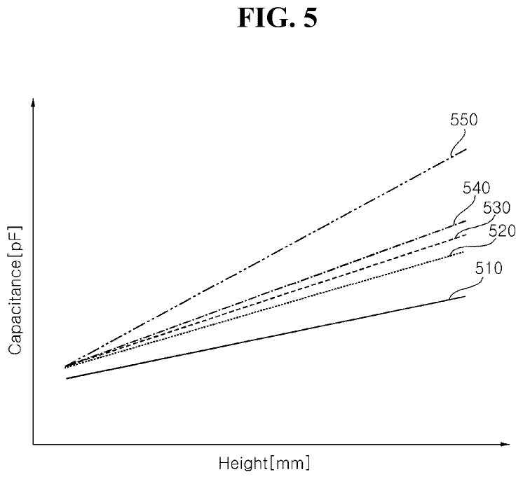

Referring to FIG. 5, as the oil surface height h1 increases, the capacitance corresponding to the plurality of upper electrodes 410, 410′ may increase. At this time, the capacitance corresponding to the plurality of upper electrodes 410, 410′ may vary depending on the concentration of oil contained in the fluid. For example, when comparing a case 510 where the oil concentration is 100%, a case 520 where the oil concentration is 75%, a case 530 where the oil concentration is 50%, a case 540 where the oil concentration is 33%, and a case 550 where the oil concentration is 0% with each other, as the oil concentration may become higher, the change in the capacitance corresponding to the plurality of upper electrodes 410, 410′ according to the oil surface height h1 may become smaller.

Therefore, since the capacitance corresponding to the plurality of upper electrodes 410, 410′ varies depending on the oil concentration, the control circuit 323 may first determine the oil concentration and then determine the height of fluid surface based on the oil concentration.

Referring again to FIG. 4, the electrode assembly 310 may include a lower electrode assembly 430. The lower electrode assembly 430 may include a first lower electrode 431 and/or a second lower electrode 435.

The first lower electrode 431 may be formed to extend horizontally. The first lower electrode 431 may be formed to have a first width w3 along the left-right direction. The first lower electrode 431 may be formed to have a second length 15 along the front-rear direction.

The first lower electrode 431 may be arranged below the plurality of upper electrodes 410, 410′. The first lower electrode 431 may be arranged to be immersed in the fluid stored inside the compressor 1. That is, in the case where a minimum amount of fluid is stored inside the compressor 1, the surface 400 of the fluid may be located above the first lower electrode 431. The first lower electrode 431 may be referred to as a first horizontal electrode 431.

The second lower electrode 435 may be formed to extend horizontally. The second lower electrode 435 may be formed to have a second width w5 that is different from the first width w3. Alternatively, the first width w3 and the second width w5 may be the same. In this disclosure, it is illustrated that the first width w3 is smaller than the second width w5. The second lower electrode 435 may be formed to have a second length 15 along the front-rear direction. The second lower electrode 435 may be referred to as a second horizontal electrode 435.

The second lower electrode 435 may be arranged below the plurality of upper electrodes 410, 410′. The second lower electrode 435 may be arranged to be immersed in the fluid stored inside the compressor 1. That is, in the case where a minimum amount of fluid is stored inside the compressor 1, the surface 400 of the fluid may be located above the second lower electrode 435.

The first lower electrode 431 and the second lower electrode 435 may be arranged in parallel along the horizontal direction. The first lower electrode 431 and the second lower electrode 435 may be arranged spaced apart from each other.

The electrode assembly 310 may include a shield electrode 420. The shield electrode 420 may be formed to extend horizontally. The shield electrode 420 may be formed to have a third width w2. The third width w2 may be greater than or equal to the sum of the first width w3 and the second width w5. The shield electrode 420 may be formed to have a third length 12 along the front-rear direction. The third length 12 may be greater than or equal to a second length 15. In the present disclosure, it is described that both ends of the shield electrode 420 correspond to the first lower electrode 431 and the second lower electrode 435, respectively, but is not limited thereto. For example, the left end of the shield electrode 420 may extend away from the first lower electrode 431. For example, the right end of the shield electrode 420 may extend away from the second lower electrode 435.

The shield electrode 420 may be arranged below the plurality of upper electrodes 410, 410′. The shield electrode 420 may be arranged so as to cover the first lower electrode 431 from the upper side. The shield electrode 420 may be arranged so as to cover the second lower electrode 435 from the upper side.

As the shield electrode 420 is arranged above the first lower electrode 431 so as to cover the first lower electrode 431, the influence of the second upper electrode 410′ on the first lower electrode 431 may be reduced. In addition, as the shield electrode 420 is arranged above the second lower electrode 435 so as to cover the second lower electrode 435, the influence of the second upper electrode 410′ on the second lower electrode 435 may be reduced.

Meanwhile, as the shield electrode 420 is arranged above the first lower electrode 431 so as to cover the first lower electrode 431, the influence of the change in the fluid level height h1 according to the amount of fluid on the capacitance corresponding to the first lower electrode 431 and the shield electrode 420 may be reduced.

In addition, as the shield electrode 420 is arranged above the second lower electrode 435 so as to cover the second lower electrode 435, the influence of the change in the fluid level height h1 according to the amount of fluid on the capacitance corresponding to the second lower electrode 435 and the shield electrode 420 may be reduced.

The shield electrode 420 may be arranged so that at least a portion of the shield electrode 420 is immersed in the fluid stored inside the compressor 1. In the case where a minimum amount of fluid is stored inside the compressor 1, the surface 400 of the fluid may be located above the lower surface of the shield electrode 420. That is, the shield electrode 420 may be arranged so that the lower surface of the shield electrode 420 is immersed in the fluid stored inside the compressor 1.

A sensing voltage may be applied to the first lower electrode 431. A sensing voltage may be applied to the second lower electrode 435. A ground voltage may be applied to the shield electrode 420. In this case, the shield electrode 420 may be referred to a ground electrode 420. Alternatively, no voltage may be applied to the shield electrode 420, the shield electrode 420 may provide passive shielding. Alternatively, a sensing voltage may be applied to the shield electrode 420, the shield electrode 420 may provide active shielding.

Since the lower surfaces of the first lower electrode 431 and the shield electrode 420 are immersed in the fluid, the capacitance corresponding to the first lower electrode 431 and the shield electrode 420 may correspond to the concentration of oil contained in the fluid. Since the lower surfaces of the second lower electrode 435 and the shield electrode 420 are immersed in the fluid, the capacitance corresponding to the second lower electrode 435 and the shield electrode 420 may correspond to the concentration of oil contained in the fluid.

The capacitance corresponding to the first lower electrode 431 and the shield electrode 420 may correspond to the oil concentration for a first depth d1 of the fluid facing downward. The capacitance corresponding to the second lower electrode 435 and the shield electrode 420 may correspond to the oil concentration for a second depth d2 of the fluid facing downward. At this time, since the first width w3 of the first lower electrode 431 and the second width w5 of the second lower electrode 435 are different, the first depth d1 and the second depth d2 may be different from each other. Since the first width w3 is smaller than the second width w5, the first depth d1 may be smaller than the second depth d2.

According to an embodiment, the control circuit 323 may determine the oil concentration corresponding to the depth of the fluid. For example, the control circuit 323 may determine the oil concentration corresponding to the first depth d1 of the fluid, based on the capacitance corresponding to the first lower electrode 431 and the shield electrode 420. For example, the control circuit 323 may determine the oil concentration corresponding to the second depth d2 of the fluid, based on the capacitance corresponding to the second lower electrode 435 and the shield electrode 420.

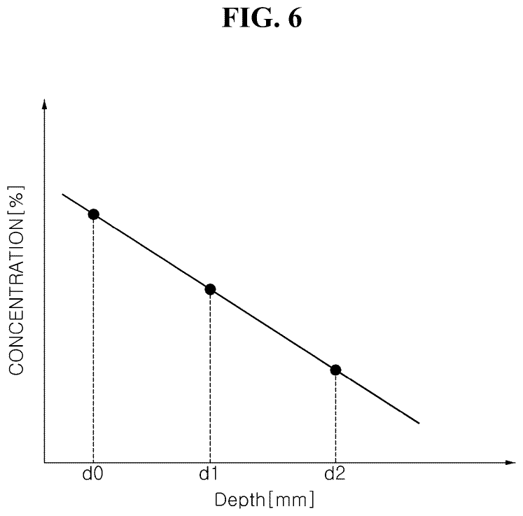

Referring to FIG. 6, when the first concentration of oil corresponding to the first depth d1 of the fluid and the second concentration of oil corresponding to the second depth d2 of the fluid are different from each other, the control circuit 323 may determine the concentration of oil corresponding to the surface d0 of the fluid, based on the first concentration of oil and the second concentration of oil. For example, the control circuit 323 may determine the concentration of oil corresponding to the surface d0 of the fluid, based on the difference between the first concentration and the second concentration, for the difference between the first depth d1 and the second depth d2. For example, the control circuit 323 may determine the concentration of oil corresponding to the surface d0 of the fluid, based on the data related to the concentration of oil corresponding to the surface d0 of the fluid, corresponding to the first concentration, the second concentration, and the difference between the first concentration and the second concentration.

Therefore, according to various embodiments of the present disclosure, the control circuit 323 may first determine the concentration of oil, and then determine the height of fluid surface based on the concentration of oil. For example, the control circuit 323 may determine the concentration of oil, based on the capacitance corresponding to the first lower electrode 431 and the shield electrode 420, and then determine the amount of fluid based on the determined concentration of oil. For example, the control circuit 323 may determine the concentration of oil, based on the capacitance corresponding to the second lower electrode 435 and the shield electrode 420, and then determine the height of fluid surface based on the determined concentration of oil. For example, the control circuit 323 may determine the concentration of oil corresponding to the surface d0 of the fluid, based on the capacitance corresponding to the first lower electrode 431 and the shield electrode 420 and the capacitance corresponding to the second lower electrode 435 and the shield electrode 420, and then determine the height of fluid surface based on the determined concentration of oil.

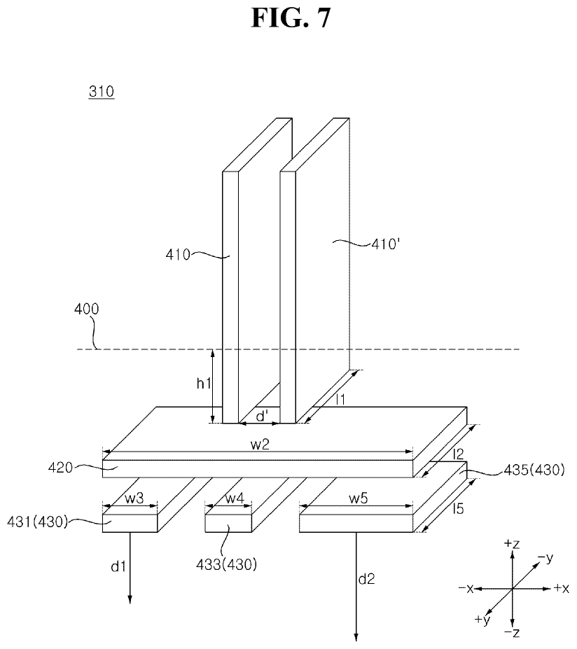

FIG. 7 is a drawing illustrating the configuration of an electrode assembly, according to another embodiment of the present disclosure. Detailed descriptions of contents overlapping with those described in FIG. 4 will be omitted.

Referring to FIG. 7, the electrode assembly 310 may further include a ground electrode 433. The ground electrode 433 may be formed to extend horizontally. The ground electrode 433 may be formed to have a fourth width w4 along the left-right direction. The ground electrode 433 may be formed to have a second length 15 along the front-rear direction.

The ground electrode 433 may be arranged below the plurality of upper electrodes 410, 410′. The ground electrode 433 may be arranged below the shield electrode 420. The ground electrode 433 may be arranged to be covered by the shield electrode 420. The ground electrode 433 may be arranged to be immersed in the fluid stored inside the compressor 1. That is, in the case where a minimum amount of fluid is stored inside the compressor 1, the surface 400 of the fluid may be located above the ground electrode 433.

The first lower electrode 431, the second lower electrode 435, and the ground electrode 433 may be arranged in parallel along the horizontal direction. The first lower electrode 431, the second lower electrode 435, and the ground electrode 433 may be arranged spaced apart from each other. The ground electrode 433 may be arranged between the first lower electrode 431 and the second lower electrode 435.

A sensing voltage may be applied to the first lower electrode 431. A sensing voltage may be applied to the second lower electrode 435. A ground voltage may be applied to the ground electrode 433.

Since the first lower electrode 431 and the ground electrode 433 are immersed in the fluid, the capacitance corresponding to the first lower electrode 431 and the ground electrode 433 may correspond to the concentration of oil contained in the fluid.

Since the second lower electrode 435 and the ground electrode 433 are immersed in the fluid, the capacitance corresponding to the second lower electrode 435 and the ground electrode 433 may correspond to the concentration of oil contained in the fluid.

The capacitance corresponding to the first lower electrode 431 and the ground electrode 433 may correspond to the concentration of oil for a third depth d3 of the fluid facing downward. The capacitance corresponding to the second lower electrode 435 and the ground electrode 433 may correspond to the concentration of oil for a fourth depth d4 of the fluid facing downward. At this time, since the first width w3 of the first lower electrode 431 and the second width w5 of the second lower electrode 435 are different, the third depth d3 and the fourth depth d4 may be different from each other. Since the first width w3 is smaller than the second width w5, the third depth d3 may be smaller than the fourth depth d4.

According to an embodiment, the control circuit 323 may determine the concentration of oil corresponding to the depth of the fluid. For example, the control circuit 323 may determine the concentration of oil corresponding to the third depth d3 of the fluid, based on the capacitance corresponding to the first lower electrode 431 and the ground electrode 433. For example, the control circuit 323 may determine the concentration of oil corresponding to the fourth depth d4 of the fluid, based on the capacitance corresponding to the second lower electrode 435 and the ground electrode 433.

When the third concentration of oil corresponding to the third depth d3 of the fluid and the fourth concentration of oil corresponding to the fourth depth d4 of the fluid are different from each other, the control circuit 323 may determine the concentration of oil corresponding to the surface d0 of the fluid, based on the third concentration of oil and the fourth concentration of oil.

Therefore, according to various embodiments of the present disclosure, the control circuit 323 may first determine the concentration of oil, and then determine the height of fluid surface based on the concentration of oil. For example, the control circuit 323 may determine the concentration of oil based on the capacitance corresponding to the first lower electrode 431 and the ground electrode 433, and then determine the height of fluid surface based on the determined concentration of oil. For example, the control circuit 323 may determine the concentration of oil based on the capacitance corresponding to the second lower electrode 435 and the ground electrode 433, and then determine the height of fluid surface based on the determined concentration of oil. For example, the control circuit 323 may determine the concentration of oil corresponding to the surface d0 of the fluid based on the capacitance corresponding to the first lower electrode 431 and the ground electrode 433 and the capacitance corresponding to the second lower electrode 435 and the ground electrode 433, and then determine the height of the fluid surface based on the determined oil concentration.

According to an embodiment, no voltage may be applied to the shield electrode 420. That is, the shield electrode 420 may be configured for passive shielding. If the shield electrode 420 is configured for passive shielding, the influence of the second upper electrode 410′ on the first lower electrode 431 and/or the second lower electrode 435 may be further reduced. In addition, when the shield electrode 420 is configured for passive shielding, the influence of the change in the fluid level height h1 according to the amount of fluid on the capacitance corresponding to the first lower electrode 431 and the ground electrode 433 and/or the capacitance corresponding to the second lower electrode 435 and the ground electrode 433 may be further reduced. In addition, when the shield electrode 420 is configured for passive shielding, the electrode assembly 310 may block the influence of noise coming in from the outside.

Meanwhile, when the shield electrode 420 is configured for passive shielding, parasitic capacitance may exist between the first lower electrode 431 and the shield electrode 420. In addition, parasitic capacitance may also exist between the second lower electrode 435 and the shield electrode 420. Such a parasitic capacitance may be a factor that reduces a signal-to-noise ratio (SNR).

According to an embodiment, a sensing voltage may be applied to the shield electrode 420. That is, the shield electrode 420 may be configured for active shielding.

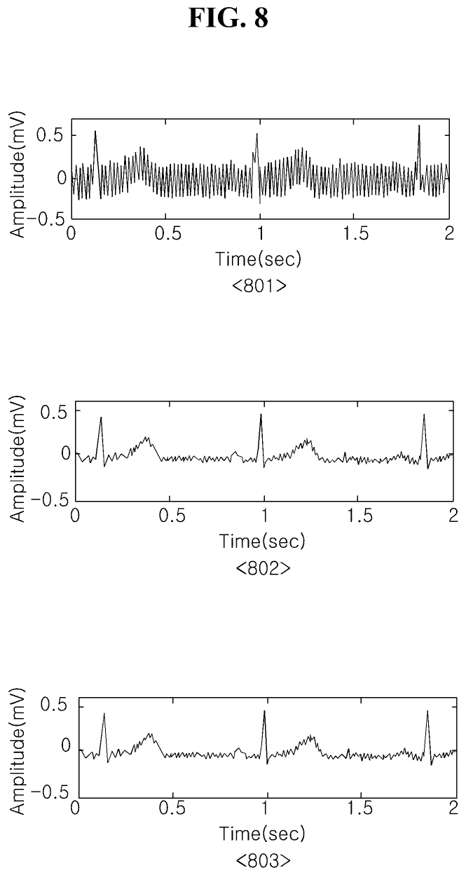

Referring to FIG. 8, when reviewing the output of the sensor in a time domain, in case 802 there is a configuration for passive shielding, or in case 803 there is a configuration for active shielding, the noise included in the output of the sensor may be significantly reduced, in comparison with a case 801 where there is no configuration for passive shielding or active shielding 801.

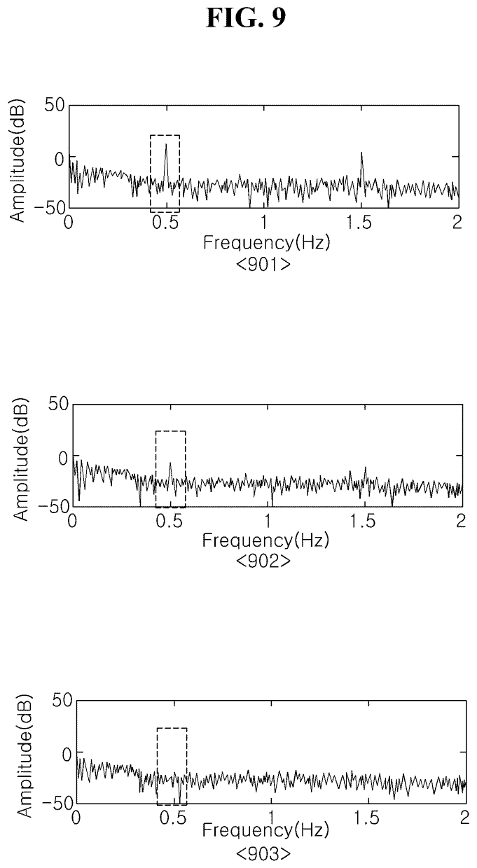

Meanwhile, referring to FIG. 9, when reviewing the output of the sensor in a frequency domain, in case 902 there is a configuration for passive shielding, the noise peak at a certain frequency (e.g., 50 Hz) may be reduced, in comparison with a case 801 where there is no configuration for passive shielding or active shielding.

In addition, in case 903 there is a configuration for active shielding, the noise peak at a certain frequency (e.g., 50 Hz) may be further reduced, in comparison with a case 902 where there is a configuration for passive shielding.

Thus, when a sensing voltage is applied to the shield electrode 420, no potential difference occurs between the first lower electrode 431 and the shield electrode 420 and between the second lower electrode 435 and the shield electrode 420, so that the parasitic capacitance can be removed. Therefore, due to the removal of the parasitic capacitance, the signal-to-noise ratio (SNR) can be improved.

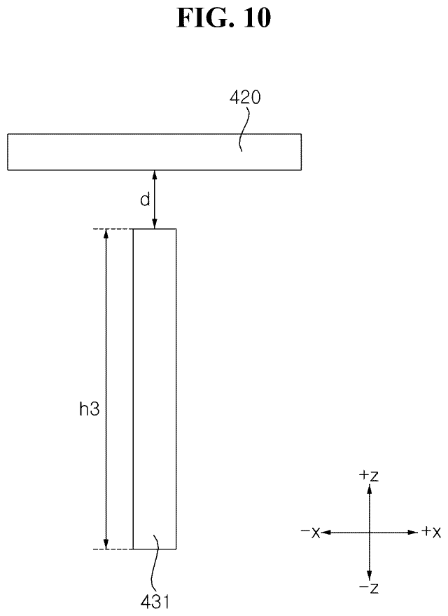

FIG. 10 to FIG. 13 are diagrams illustrating a configuration of an electrode assembly, according to embodiments of the present disclosure.

Referring to FIG. 10 to FIG. 13, the lower electrode assembly 430 may be disposed below the shield electrode 420. The upper electrode assembly 410, 410′ may be disposed above the shield electrode 420 (see FIGS. 4 and 7). As the lower electrode assembly 430 is disposed on the lower side of the shield electrode 420, the lower electrode assembly 430 may be disposed to be immersed in the fluid stored inside the compressor 1. That is, in the case where a minimum amount of fluid is stored inside the compressor 1, the fluid surface 400 may be located above the lower electrode assembly 430.

The lower electrode assembly 430 may be disposed to correspond to the shield electrode 420. The shield electrode 420 may be disposed to cover the lower electrode assembly 430 from above. That is, in the horizontal direction, the size of the lower electrode assembly 430 may be smaller than the size of the shield electrode 420. For example, when viewing the lower surface of the shield electrode 420 in the upper direction, the lower electrode assembly 430 may be located inside the shield electrode 420.

As the shield electrode 420 is disposed on the upper side of the lower electrode assembly 430 to cover the lower electrode assembly 430, the influence of the upper electrode 410 on the lower electrode assembly 430 may be reduced. In addition, as the shield electrode 420 is disposed on the upper side of the lower electrode assembly 430 to cover the lower electrode assembly 430, the influence of the change in the height of the fluid surface 400 on the lower electrode assembly 430 may be reduced.

The lower electrode assembly 430 may include at least one lower electrode to which a sensing voltage is applied. The lower electrode assembly 430 may further include a ground electrode 433 to which a ground voltage is applied.

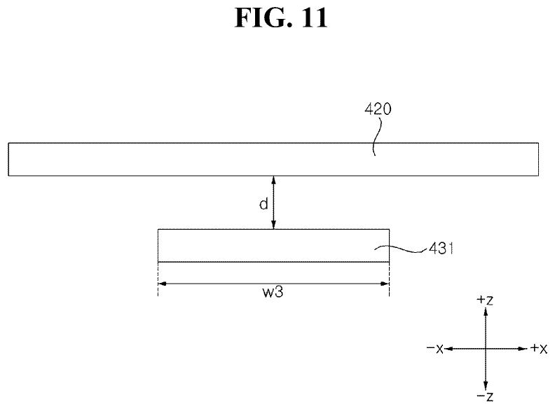

Referring to FIGS. 10 and 11, the lower electrode assembly 430 may include a first lower electrode 431.

As shown in FIG. 10, the first lower electrode 431 may be formed to extend vertically. The first lower electrode 431 may be formed to have a third height h3 along the up-down direction. The first lower electrode 431 may be formed to have a third length 13 along the front-rear direction.

Meanwhile, as shown in FIG. 11, the first lower electrode 431 may be formed to extend horizontally. The first lower electrode 431 may be formed to have a third width w3 along the left-right direction. The first lower electrode 431 may be formed to have a third length 13 along the front-rear direction.

The capacitance corresponding to the first lower electrode 431 may be detected using a self-capacitance method. At this time, since the first lower electrode 431 is always immersed in the fluid, the capacitance corresponding to the first lower electrode 431 may correspond to the concentration of oil contained in the fluid. That is, the sensor 300 may determine the concentration of oil contained in the fluid, based on the capacitance corresponding to the first lower electrode 431.

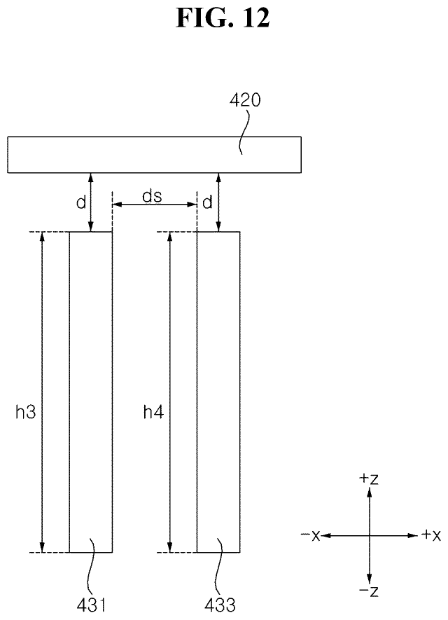

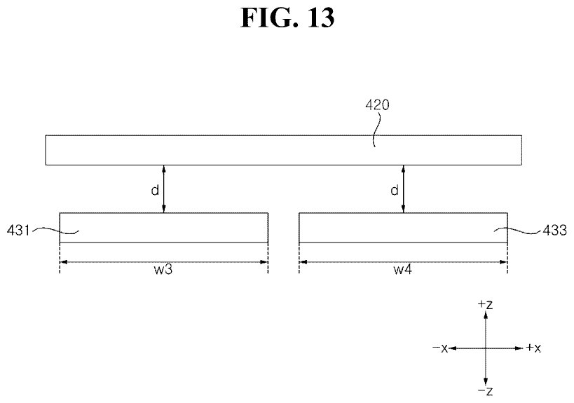

Referring to FIGS. 12 and 13, the lower electrode assembly 430 may include a ground electrode 433.

As shown in FIG. 12, the ground electrode 433 may be formed to extend vertically. The ground electrode 433 may be formed to have a fourth height h4 along the up-down direction. The ground electrode 433 may be formed to have a fourth length 14 along the front-rear direction. For example, the fourth height h4 of the ground electrode 433 may correspond to the third length h3 of the first lower electrode 431. For example, the fourth length 14 of the ground electrode 433 may correspond to the third length 13 of the first lower electrode 431.

Meanwhile, as shown in FIG. 13, the first lower electrode 431 may be formed to extend horizontally. The ground electrode 433 may be formed to have a fourth width w4 along the left-right direction.

The capacitance corresponding to the first lower electrode 431 may be detected by using a mutual capacitance method for the first lower electrode 431 and the ground electrode 433. At this time, since the first lower electrode 431 and the ground electrode 433 are always immersed in the fluid, the capacitance corresponding to the first lower electrode 431 may correspond to the concentration of oil contained in the fluid. That is, the sensor 300 may determine the concentration of oil contained in the fluid, based on the capacitance corresponding to the first lower electrode 431.

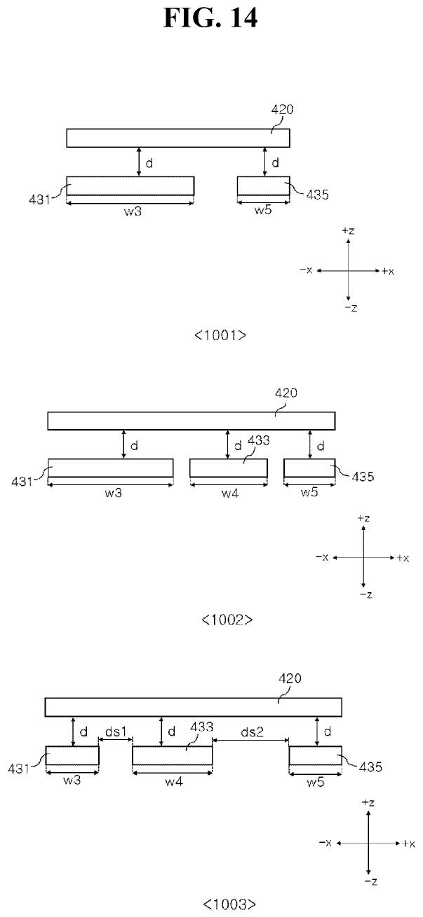

FIG. 14 to FIG. 21 are diagrams illustrating a configuration of an electrode assembly, according to embodiments of the present disclosure.

Referring to FIGS. 14 to 21, depending on the shape, size, position, and distance from other configurations of each of the first lower electrode 431 and the second lower electrode 435 according to embodiments of the present disclosure, the capacitance corresponding to the first lower electrode 431 and the capacitance corresponding to the second lower electrode 435 may be different from each other. At this time, the difference between the capacitance corresponding to the first lower electrode 431 and the capacitance corresponding to the second lower electrode 435 may correspond to the difference in oil concentration for the depth of the fluid directed downward. Accordingly, the sensor 300 may determine the oil concentration corresponding to the depth of the fluid, based on the capacitance corresponding to the first lower electrode 431, the capacitance corresponding to the second lower electrode 435, and/or the difference between the two capacitances.

Referring to FIG. 14, the lower electrode assembly 430 may include the first lower electrode 431 and the second lower electrode 435. The first lower electrode 431 and the second lower electrode 435 may be formed to extend horizontally.

The first lower electrode 431 and the second lower electrode 435 may be disposed to be spaced apart from the shield electrode 420 in the vertical direction by a certain distance d. The first lower electrode 431 and the second lower electrode 435 may be disposed side by side along the horizontal direction.

Referring to the reference numeral 1001 of FIG. 14, the third width w3 of the first lower electrode 431 may be different from the fifth width w5 of the second lower electrode 435.

The capacitance corresponding to the first lower electrode 431 and the capacitance corresponding to each second lower electrode 435 may be detected by using the self-capacitance method. At this time, since the third width w3 of the first lower electrode 431 and the fifth width w5 of the second lower electrode 435 are different, the capacitance corresponding to the first lower electrode 431 and the capacitance corresponding to the second lower electrode 435 may be different.

Referring to reference numeral 1002 of FIG. 14, the third width w3 of the first lower electrode 431 may be different from the fifth width w5 of the second lower electrode 435.

A ground electrode 433 may be disposed between the first lower electrode 431 and the second lower electrode 435.

The capacitance corresponding to the first lower electrode 431 and the capacitance corresponding to each second lower electrode 435 may be detected by using the mutual capacitance method for the ground electrode 433. At this time, since the third width w3 of the first lower electrode 431 and the fifth width w5 of the second lower electrode 435 are different, the capacitance corresponding to the first lower electrode 431 and the capacitance corresponding to the second lower electrode 435 may be different.

Referring to reference numeral 1003 of FIG. 14, the third width w3 of the first lower electrode 431 may correspond to the fifth width w5 of the second lower electrode 435.

The ground electrode 433 may be disposed between the first lower electrode 431 and the second lower electrode 435. In the horizontal direction, the distance ds1 between the first lower electrode 431 and the ground electrode 433 and the distance ds2 between the second lower electrode 435 and the ground electrode 433 may be different.

The capacitance corresponding to the first lower electrode 431 and the capacitance corresponding to each second lower electrode 435 may be detected by using the mutual capacitance method for the ground electrode 433. At this time, since the distance ds1 between the first lower electrode 431 and the ground electrode 433 and the distance ds2 between the second lower electrode 435 and the ground electrode 433 are different, the capacitance corresponding to the first lower electrode 431 and the capacitance corresponding to the second lower electrode 435 may be different.

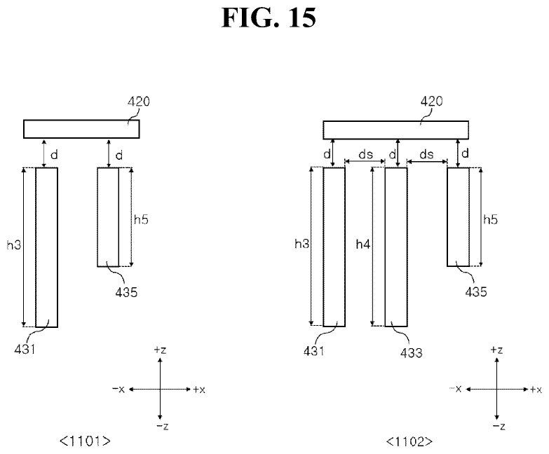

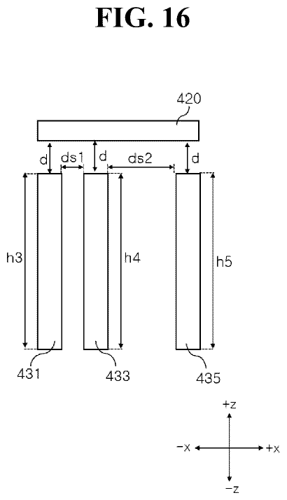

Referring to FIGS. 15 and 16, the lower electrode assembly 430 may include the first lower electrode 431 and the second lower electrode 435. The first lower electrode 431 and the second lower electrode 435 may be formed to extend vertically.

The first lower electrode 431 and the second lower electrode 435 may be disposed to be spaced apart from the shield electrode 420 in the vertical direction by a certain distance d. The first lower electrode 431 and the second lower electrode 435 may be disposed side by side along the horizontal direction.

Referring to reference numeral 1101 of FIG. 15, the third height h3 of the first lower electrode 431 may be different from the fifth height h5 of the second lower electrode 435.

The capacitance corresponding to the first lower electrode 431 and the capacitance corresponding to each second lower electrode 435 may be detected by using the self-capacitance method. At this time, since the third height h3 of the first lower electrode 431 and the fifth height h5 of the second lower electrode 435 are different, the capacitance corresponding to the first lower electrode 431 and the capacitance corresponding to the second lower electrode 435 may be different.

Referring to reference numeral 1102 of FIG. 15, the third height h3 of the first lower electrode 431 may be different from the fifth height h5 of the second lower electrode 435.

A ground electrode 433 may be disposed between the first lower electrode 431 and the second lower electrode 435.

The capacitance corresponding to the first lower electrode 431 and the capacitance corresponding to each second lower electrode 435 may be detected by using the mutual capacitance method for the ground electrode 433. At this time, since the third height h3 of the first lower electrode 431 and the fifth height h5 of the second lower electrode 435 are different, the capacitance corresponding to the first lower electrode 431 and the capacitance corresponding to the second lower electrode 435 may be different.

Referring to FIG. 16, the third height h3 of the first lower electrode 431 may correspond to the fifth height h5 of the second lower electrode 435.

The ground electrode 433 may be disposed between the first lower electrode 431 and the second lower electrode 435. In the horizontal direction, the distance ds1 between the first lower electrode 431 and the ground electrode 433 and the distance ds2 between the second lower electrode 435 and the ground electrode 433 may be different.

The capacitance corresponding to the first lower electrode 431 and the capacitance corresponding to each second lower electrode 435 may be detected by using the mutual capacitance method for the ground electrode 433. At this time, since the distance ds1 between the first lower electrode 431 and the ground electrode 433 and the distance ds2 between the second lower electrode 435 and the ground electrode 433 are different, the capacitance corresponding to the first lower electrode 431 and the capacitance corresponding to the second lower electrode 435 may be different.

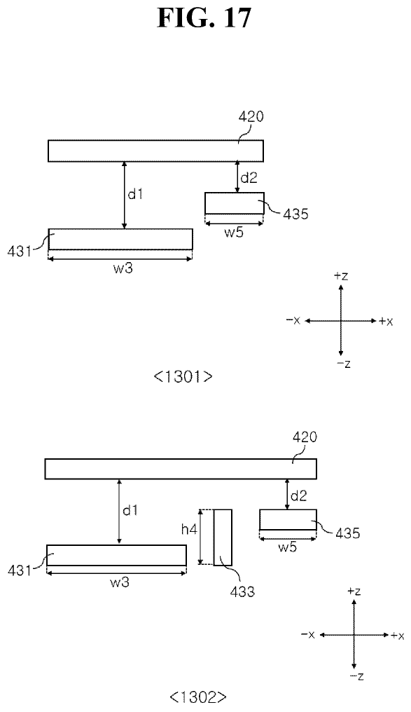

Referring to FIG. 17, the lower electrode assembly 430 may include the first lower electrode 431 and the second lower electrode 435. The first lower electrode 431 and the second lower electrode 435 may be formed to extend horizontally.

The third width w3 of the first lower electrode 431 and the fifth width w5 of the second lower electrode 435 may correspond to each other, or may be different.

The first lower electrode 431 may be disposed to be spaced apart from the shield electrode 420 in the vertical direction by a first distance d1. The second lower electrode 435 may be disposed to be spaced apart from the shield electrode 420 in the vertical direction by a second distance d2. That is, the depth of the fluid directed downward, corresponding to the positions where the first lower electrode 431 and the second lower electrode 435 are respectively disposed, may be different.

Referring to reference numeral 1301, the capacitance corresponding to the first lower electrode 431 and the capacitance corresponding to the second lower electrode 435 may be detected by using a self-capacitance method. At this time, since the depth of the fluid corresponding to the positions where the first lower electrode 431 and the second lower electrode 435 are respectively disposed are different, the capacitance corresponding to the first lower electrode 431 and the capacitance corresponding to the second lower electrode 435 may be different.

Referring to reference numeral 1302, a ground electrode 433 may be disposed between the first lower electrode 431 and the second lower electrode 435 in the horizontal direction.

When the first distance d1 between the first lower electrode 431 and the shield electrode 420 exceeds the second distance d2 between the second lower electrode 435 and the shield electrode 420, the fourth height h4 of the ground electrode 433 may be greater than or equal to the distance between the lower surface of the first lower electrode 431 and the upper surface of the second lower electrode 435.

The capacitance corresponding to the first lower electrode 431 and the capacitance corresponding to each second lower electrode 435 may be detected by using a mutual capacitance method for the ground electrode 433. At this time, since the depth of the fluid corresponding to the positions where the first lower electrode 431 and the second lower electrode 435 are respectively disposed is different, the capacitance corresponding to the first lower electrode 431 and the capacitance corresponding to the second lower electrode 435 may be different.

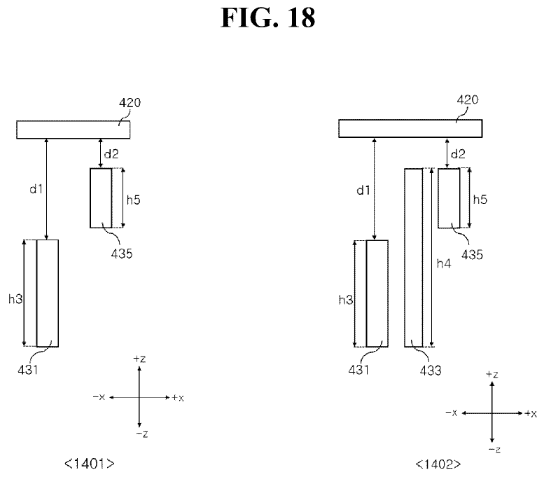

Referring to FIG. 18, the lower electrode assembly 430 may include the first lower electrode 431 and the second lower electrode 435. The first lower electrode 431 and the second lower electrode 435 may be formed to extend vertically.

The third height h3 of the first lower electrode 431 and the fifth height h5 of the second lower electrode 435 may correspond to each other, or may be different.

The first lower electrode 431 may be disposed to be spaced apart from the shield electrode 420 in the vertical direction by a first distance d1. The second lower electrode 435 may be disposed to be spaced apart from the shield electrode 420 in the vertical direction by a second distance d2. That is, the depth of the fluid directed downward, corresponding to the positions where the first lower electrode 431 and the second lower electrode 435 are respectively disposed, may be different.

Referring to reference numeral 1401, the capacitance corresponding to the first lower electrode 431 and the capacitance corresponding to the second lower electrode 435 may be detected by using a self-capacitance method. At this time, since the depth of the fluid corresponding to the positions where the first lower electrode 431 and the second lower electrode 435 are respectively disposed are different, the capacitance corresponding to the first lower electrode 431 and the capacitance corresponding to the second lower electrode 435 may be different.

Referring to reference numeral 1402, a ground electrode 433 may be disposed between the first lower electrode 431 and the second lower electrode 435 in the horizontal direction.

When the first distance d1 between the first lower electrode 431 and the shield electrode 420 exceeds the second distance d2 between the second lower electrode 435 and the shield electrode 420, the fourth height h4 of the ground electrode 433 may be greater than or equal to the distance between the lower surface of the first lower electrode 431 and the upper surface of the second lower electrode 435.

The capacitance corresponding to the first lower electrode 431 and the capacitance corresponding to each second lower electrode 435 may be detected by using a mutual capacitance method for the ground electrode 433. At this time, since the depth of the fluid corresponding to the positions where the first lower electrode 431 and the second lower electrode 435 are respectively disposed is different, the capacitance corresponding to the first lower electrode 431 and the capacitance corresponding to the second lower electrode 435 may be different.

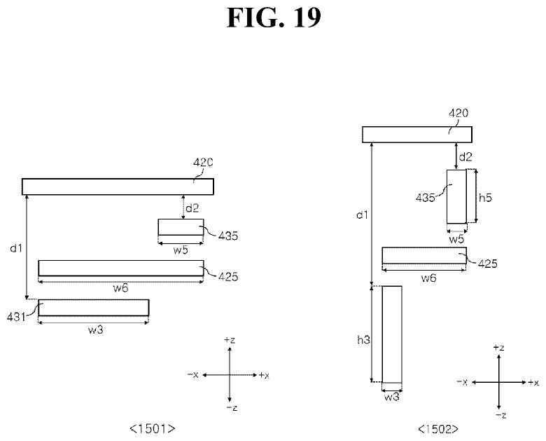

Referring to FIG. 19, the lower electrode assembly 430 may include the first lower electrode 431 and the second lower electrode 435. The first lower electrode 431 and the second lower electrode 435 may be formed to extend horizontally.

The third width w3 of the first lower electrode 431 and the fifth width w5 of the second lower electrode 435 may correspond to each other, or may be different. The third height h3 of the first lower electrode 431 and the fifth height h5 of the second lower electrode 435 may correspond to each other, or may be different.

The first lower electrode 431 may be disposed vertically apart from the shield electrode 420 by a first distance d1. The second lower electrode 435 may be disposed vertically apart from the shield electrode 420 by a second distance d2. That is, the depths of the fluids directed downward, corresponding to the positions where the first lower electrode 431 and the second lower electrode 435 are disposed, may be different.

The electrode assembly 310 may further include a sub-shield electrode 425. The sub-shield electrode 425 may be formed to extend horizontally.

The sub-shield electrode 425 may be formed to have a sixth width w6 along the left-right direction. When the first lower electrode 431 is disposed on the left side and the second lower electrode 435 is disposed on the right side, the sixth width w6 of the sub-shield electrode 425 may be greater than or equal to the distance between the left surface of the first lower electrode 431 and the right surface of the second lower electrode 435.

The sub-shield electrode 425 may be formed to have a sixth length along the front-rear direction. The sixth length of the sub-shield electrode 425 may correspond to the third length 13 of the first lower electrode 431 and the fifth length 15 of the second lower electrode 435.

According to an embodiment, a sensing voltage may be applied to the sub-shield electrode 425. That is, the sub-shield electrode 425 may be configured for active shielding.

The capacitance corresponding to the first lower electrode 431 and the capacitance corresponding to each second lower electrode 435 may be detected by using a self-capacitance method. At this time, since the depth of the fluid corresponding to the positions where the first lower electrode 431 and the second lower electrode 435 are disposed respectively is different, the capacitance corresponding to the first lower electrode 431 and the capacitance corresponding to the second lower electrode 435 may be different.

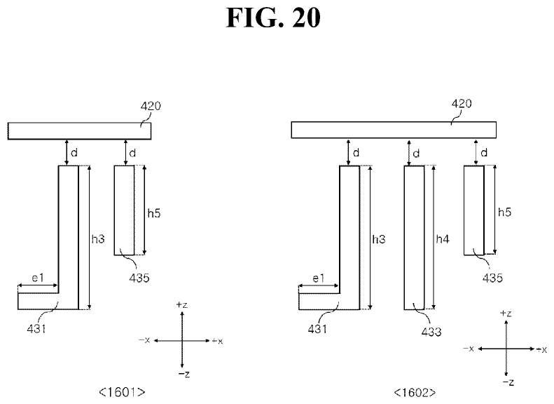

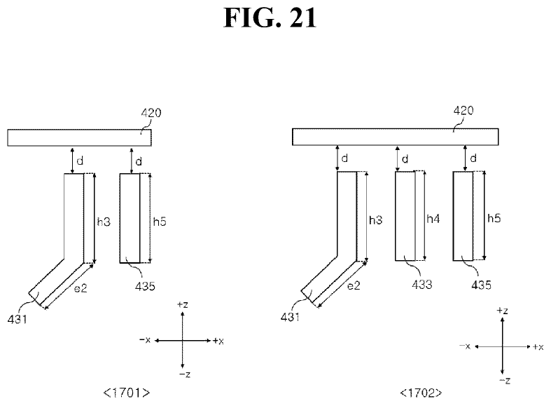

Referring to FIGS. 20 and 21, the lower electrode assembly 430 may include the first lower electrode 431 and the second lower electrode 435. The first lower electrode 431 and the second lower electrode 435 may be formed to extend vertically.

The third height h3 of the first lower electrode 431 and the fifth height h5 of the second lower electrode 435 may correspond to each other, or may be different.

The first lower electrode 431 and the second lower electrode 435 may be disposed vertically apart from the shield electrode 420 by a certain distance d. The first lower electrode 431 and the second lower electrode 435 may be disposed side by side along the horizontal direction.

The shape of the first lower electrode 431 and the shape of the second lower electrode 435 may be different. The shape of the first lower electrode 431 and the shape of each second lower electrode 435 may correspond to the surrounding environment in which the sensor 300 is disposed, the internal structure of the sensor 300 in which the lower electrode assembly 430 is disposed, etc.

For example, referring to FIG. 20, the lower one end of the first lower electrode 431 may be formed to extend in the left direction by a first extension length e1. For example, referring to FIG. 17, the first lower electrode 431 may be formed to extend in the left direction and the lower direction by a second extension length e2.

Referring to the reference numeral 1601 of FIG. 20 and the reference numeral 1701 of FIG. 21, the capacitance corresponding to the first lower electrode 431 and the capacitance corresponding to each second lower electrode 435 may be detected by using a self-capacitance method. At this time, since the shape of the first lower electrode 431 and the shape of the second lower electrode 435 are different, the capacitance corresponding to the first lower electrode 431 and the capacitance corresponding to the second lower electrode 435 may be different.

Referring to the reference numeral 1602 of FIG. 20 and the reference numeral 1702 of FIG. 21, a ground electrode 433 may be disposed between the first lower electrode 431 and the second lower electrode 435.

The capacitance corresponding to the first lower electrode 431 and the capacitance corresponding to each second lower electrode 435 may be detected by using a mutual capacitance method for the ground electrode 433. At this time, since the shape of the first lower electrode 431 and the shape of the second lower electrode 435 are different, the capacitance corresponding to the first lower electrode 431 and the capacitance corresponding to the second lower electrode 435 may be different.

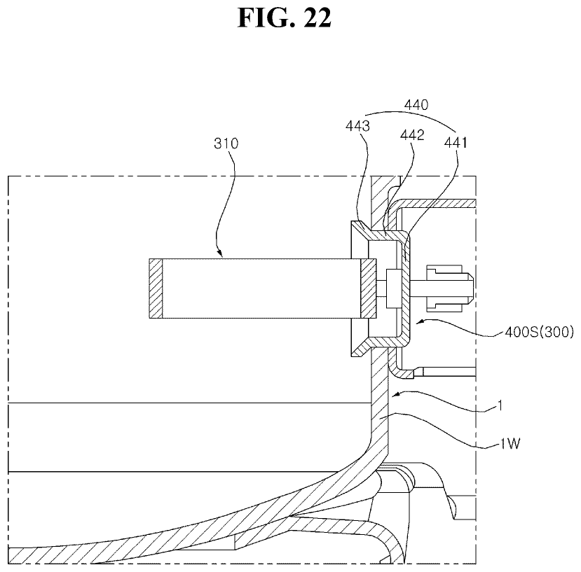

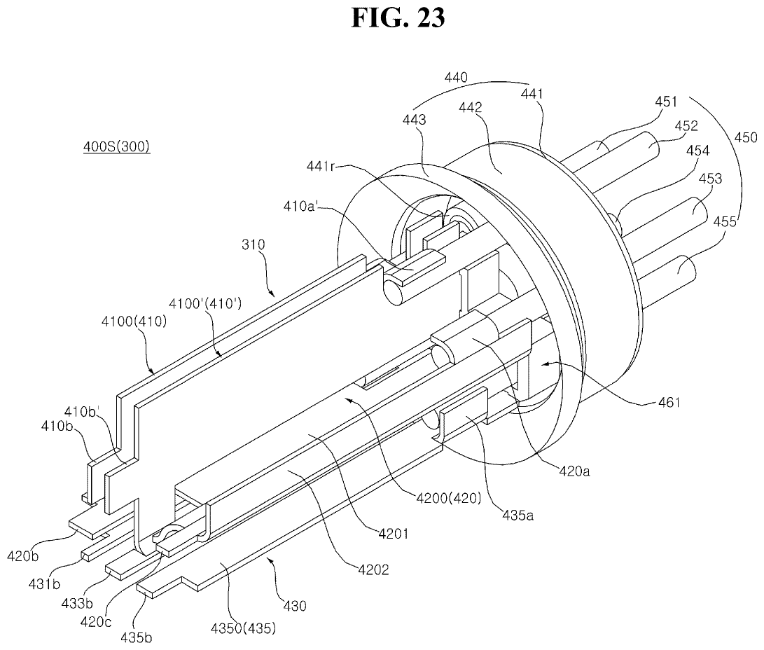

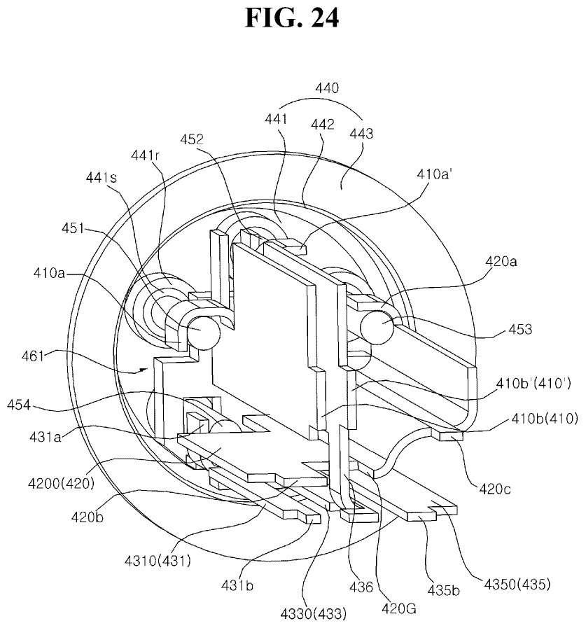

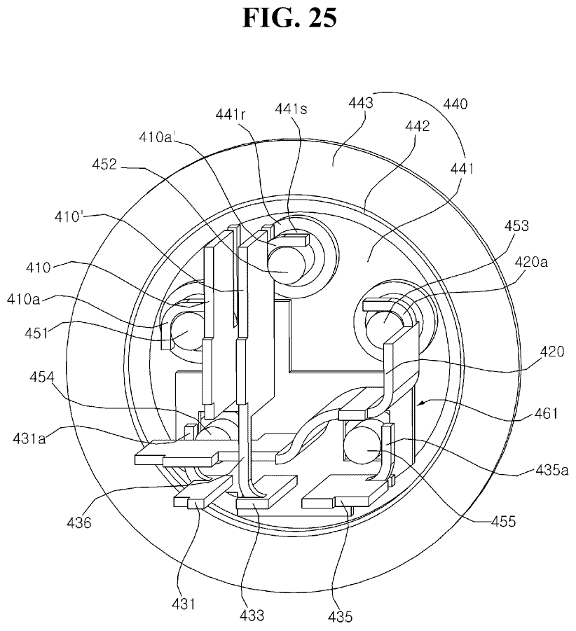

FIGS. 22 to 28 are diagrams illustrating a sensing module of a sensor according to an embodiment of the present disclosure.

Referring to FIGS. 22 to 25, the sensor 300 may include a sensing module 400S. The sensing module 400S may include the electrode assembly 310, a housing 440, and a pin assembly 450.

For example, the electrode assembly 310 may be installed in a lower portion of the compressor 1 (see FIGS. 1A and 1B). The electrode assembly 310 may be inserted into the compressor 1, and the pin assembly 450 may be electrically connected to the processing circuit 320 (see FIG. 3) outside the compressor 1 via a cable.

The housing 440 may have an overall cap shape. The housing 440 may be referred to as a cap 440. The housing 440 may be opened toward the electrode assembly 310. A bottom 441 of the housing 440 may have a disc shape. Alternatively, the bottom 441 of the housing 440 may have an angular shape (for example, a square or a pentagon). A side wall 442 of the housing 440 may extend along an edge of the bottom 441 of the housing 440. A flange 443 of the housing 440 may protrude from a side surface of the side wall 442 and may extend along the side wall 442. The diameter (width) of the flange 443 of the housing 440 may be larger than the diameter (width) of the side wall 442 of the housing 440. The flange 443 of the housing 440 may be adjacent to a distal end of the side wall 442. The housing 440 may penetrate a body 1W of the compressor 1. The flange 443 of the housing 440 may face an inner side surface of the body 1W of the compressor 1, and the side wall 442 of the housing 440 may be welded to the body 1W of the compressor 1.

The electrode assembly 310 may face the bottom 441 of the housing 440. The electrode assembly 310 may be spaced apart from the bottom 441 of the housing 440 and may extend in a direction intersecting the bottom 441. The electrode assembly 310 may include a material having electrical conductivity, such as metal. The electrode assembly 310 may include the upper electrode assembly 410, 410′, the shield electrode 420, and the lower electrode assembly 430. The upper electrode assembly 410, 410′ may be referred to as a liquid level detection unit 410, 410′. The lower electrode assembly 430 may be referred to as a concentration detection unit 430.

The upper electrode assembly 410, 410′ may have a plate shape extending in a vertical direction. The upper electrode assembly 410, 410′ may be spaced apart from each other in a horizontal direction. The upper electrode assembly 410, 410′ may include an upper electrode 410 and a ground electrode 410′.

The upper electrode 410 may have a vertical plate shape. The upper electrode 410 may have an overall rectangular shape. The upper electrode 410 may have two long sides that are opposite to each other and two short sides that are opposite to each other. A coupling portion 410a may be adjacent to one end of the upper electrode 410 and may protrude from a body 4100 of the upper electrode 410. The coupling portion 410a may be bent from the body 4100 of the upper electrode 410. The protrusion portion 410b may form the other end of the upper electrode 410 and may protrude from the body 4100 of the upper electrode 410.

The ground electrode 410′ may have a vertical plate shape. The ground electrode 410′ may have an overall rectangular shape. The ground electrode 410′ may have two long sides that are opposite to each other and two short sides that are opposite to each other. The coupling portion 410a may be adjacent to one end of the ground electrode 410′ and may protrude from a body 4100′ of the ground electrode 410′. A coupling portion 410a′ may be bent from the body 4100′ of the ground electrode 410′. The protrusion portion 410b′ may form the other end of the ground electrode 410′ and may protrude from the body 4100′ of the ground electrode 410′.

The shield electrode 420 may be spaced downward from the upper electrode assembly 410, 410′. The shield electrode 420 may have a plate shape extending in a direction intersecting the upper electrode assembly 410, 410′. A coupling portion 420a may be adjacent to one end of the shield electrode 420 and may protrude from a body 4200 of the shield electrode 420. The coupling portion 420a may be bent from the body 4200 of the shield electrode 420. A first protrusion portion 420b may form the other end of the shield electrode 420 and may protrude from the body 4200 of the shield electrode 420. A second protrusion portion 420c may form the other end of the shield electrode 420 and may protrude from the body 4200 of the shield electrode 420. The first protrusion portion 420b and the second protrusion portion 420c may be spaced apart from each other. The shield body 4200 may cover the lower edges of the upper electrode assembly 410, 410′. The area of the shield body 4200 may be larger than the area of the lower electrode assembly 430 described below.

The lower electrode assembly 430 may be spaced downward from the shield electrode 420. The lower electrode assembly 430 may have a plate shape extending in a direction parallel to the shield electrode 420. The lower electrode assembly 430 may be extended long in a lengthwise direction of the shield electrode 420. The electrodes of the lower electrode assembly 430 may be spaced apart from each other in a horizontal direction. The lower electrode assembly 430 may include the first lower electrode 431, the second lower electrode 435, and the ground electrode 433.

The first lower electrode 431 may have a plate shape parallel to the shield electrode 420. The first lower electrode 431 may have an overall rectangular shape. The first lower electrode 431 may have two long sides that are opposite to each other and two short sides that are opposite to each other. An upper surface of the first lower electrode 431 may be adjacent to a lower surface of the shield electrode 420. A first coupling portion 431a may be adjacent to one end of the first lower electrode 431 and may protrude from a body 4310 of the first lower electrode 431. The first coupling portion 431a may be bent from the body 4310 of the first lower electrode 431. The first protrusion portion 431b may form the other end of the first lower electrode 431 and may protrude from the body 4310 of the first lower electrode 431. The shield body 4200 may cover the upper surface of the first lower electrode 431.

The second lower electrode 435 may have a plate shape parallel to the shield electrode 420. The second lower electrode 435 may be spaced apart from the first lower electrode 431 in a horizontal direction. The second lower electrode 435 may have an overall rectangular shape. The second lower electrode 435 may have two long sides that are opposite to each other and two short sides that are opposite to each other. The upper surface of the second lower electrode 435 may be adjacent to the lower surface of the shield electrode 420. A second coupling portion 435a may be adjacent to one end of the second lower electrode 435 and may protrude from a body 4350 of the second lower electrode 435. The second coupling portion 435a may be bent from the body 4350 of the second lower electrode 435. The second protrusion portion 435b may form the other end of the second lower electrode 435 and may protrude from the body 4350 of the second lower electrode 435. The shield body 4200 may cover the upper surface of the second lower electrode 435.

The ground electrode 433 may have a plate shape parallel to the shield electrode 420. The ground electrode 433 may be disposed between the first lower electrode 431 and the second lower electrode 435, and may be horizontally spaced from the first lower electrode 431 and the second lower electrode 435. The ground electrode 433 may have an overall rectangular shape. The ground electrode 433 may have two long sides that are opposite to each other and two short sides that are opposite to each other. The upper surface of the ground electrode 433 may be adjacent to the lower surface of the shield electrode 420. The shield body 4200 may cover the upper surface of the ground electrode 433.

A bridge 436 may extend in a direction intersecting the shield electrode 420. The shield electrode 420 may include a groove 420G through which the bridge 436 passes. The groove 420G may be formed from a distal end of the shield electrode 420 to an inner side of the shield electrode 420, and the bridge 436 may be located in the groove 420G. The bridge 436 may be spaced apart from a boundary surface of the groove 420G. One end of the bridge 436 may be adjacent to the other end of the ground electrode 410′ of the upper electrode assembly 410, 410′ and may be connected to the ground electrode 410′. The other end of the bridge 436 may be adjacent to the other end of the ground electrode 433 of the lower electrode assembly 430 and may be connected to the ground electrode 433. In other words, the bridge 436 may connect the ground electrode 410′ and the ground electrode 433.

Accordingly, the shield electrode 420 may be disposed between the upper electrode assembly 410, 410′ and the lower electrode assembly 430, and may shield the upper electrode assembly 410, 410′ from the lower electrode assembly 430, while shielding the lower electrode assembly 430 from the upper electrode assembly 410, 410′.

The pin assembly 450 may penetrate the bottom 441 of the housing 440. The pin assembly 450 may include a material having electrical conductivity, such as metal. The side wall 442 of the housing 440 may surround the pin assembly 450. A rib 441r protruding from an inner side surface of the bottom 441 may surround a portion of an outer side surface of the pin assembly 450. A sealant 441s may block a gap between the outer side surface of the pin assembly 450 and the inner side surface of the rib 441r. The sealant 441s may be made of an electrically insulating material such as glass. The sealant 441s may be referred to as an insulating material 441s or a glass frit 441s.

The pin assembly 450 may include a plurality of pins. The pins may be electrically conductive. The pins may be referred to as conductive pins. The number of the pins may be equal to or less than the number of electrodes of the electrode assembly 310.

For example, the pin assembly 450 may include a first pin 451, a second pin 452, a third pin 453, a fourth pin 454, and a fifth pin 455. The first to fifth pins 451, 452, 453, 454, 455 may extend in a direction intersecting the bottom 441 of the housing 440. The first to fifth pins 451, 452, 453, 454, 455 may be disposed adjacent to an edge of the bottom 441 of the housing 440 and along the edge. In other words, the first to fifth pins 451, 452, 453, 454, 455 may be disposed radially with respect to the center of the bottom 441 of the housing 440. The first pin 451 may be electrically connected to the upper electrode 410. The second pin 452 may be electrically connected to the ground electrode 410′. The third pin 453 may be electrically connected to the shield electrode 420. The fourth pin 454 may be electrically connected to the first lower electrode 431. The fifth pin 455 may be electrically connected to the second lower electrode 435.

The sensing voltage may be applied to each of the upper electrode 410, the shield electrode 420, the first lower electrode 431 and the second lower electrode 435 through the first pin 451, the third pin 453, the fourth pin 454, and the fifth pin 455, respectively. The ground electrode may be applied to the ground electrode 410′ through the second pin 452 and to the ground electrode 433 through the bridge 436. In this connection, the shield electrode 420 may provide active shielding.

Alternatively, the third pin 453 may be omitted so that no voltage is applied to the shield electrode 420. In this connection, the shield electrode 420 may provide passive shielding.

Alternatively, the sixth pin (not shown) may be electrically connected to the ground electrode 433, and the ground electrode may be applied to the ground electrode 433 through the sixth pin. When the second pin 452 and the sixth pin are provided simultaneously, the bridge 436 may be omitted. When the second pin 452 is omitted but the sixth pin is provided, the ground electrode may be applied to the ground electrode 410′ through the bridge 436.

The first to third pins 451, 452, 453 may be disposed above the shield electrode 420, and the fourth and fifth pins 454, 455 may be disposed below the shield electrode 420.

A portion of the first pin 451 may extend from the bottom 441 of the housing 440 toward a side space of the upper electrode 410, and the remainder of the first pin 451 may extend in an opposite direction of a portion thereof. The coupling portion 410a of the upper electrode 410 may contact and be coupled to the first pin 451. The coupling portion 410a may be bent from the upper electrode 410 to surround a portion of the first pin 451 and may be welded to the first pin 451. In other words, the upper electrode 410 may be hung on the first pin 451.

A portion of the second pin 452 may extend from the bottom 441 of the housing 440 toward a side space of the ground electrode 410′, and the remainder of the second pin 452 may extend in an opposite direction of the portion thereof. A coupling portion 410a′ of the ground electrode 410′ may contact and be coupled to the second pin 452. The coupling portion 410a′ may be bent from the ground electrode 410′ to surround a portion of the second pin 452 and may be welded to the second pin 452. In other words, the ground electrode 410′ may be hung on the second pin 452.

A portion of the third pin 453 may extend from the bottom 441 of the housing 440 toward an upper space of the shield electrode 420, and the remainder of the third pin 453 may extend in an opposite direction of the portion thereof. The coupling portion 420a of the shield electrode 420 may contact and be coupled to the third pin 453. The coupling portion 420a may be bent from the shield electrode 420 to surround a portion of the third pin 453 and may be welded to the third pin 453. In other words, the shield electrode 420 may be hung on the third pin 453.

A portion of the fourth pin 454 may extend from the bottom 441 of the housing 440 toward the shield electrode 420, and the remainder of the fourth pin 454 may extend in an opposite direction of the portion thereof. The first coupling portion 431a of the first lower electrode 431 may contact and be coupled to the fourth pin 454. The first coupling portion 431a may be bent from the first lower electrode 431 to surround a portion of the fourth pin 454 and may be welded to the fourth pin 454. In other words, the first lower electrode 431 may be hung on the fourth pin 454.

A portion of the fifth pin 455 may extend from the bottom 441 of the housing 440 toward a lower space of the shield electrode 420, and the remainder of the fifth pin 455 may extend in an opposite direction of the portion thereof. The second coupling portion 435a of the second lower electrode 435 may contact and be coupled to the fifth pin 455. The second coupling portion 435a may be bent from the second lower electrode 435 to surround a portion of the fifth pin 455 and may be welded to the fifth pin 455. In other words, the second lower electrode 435 may be hung on the fifth pin 455.

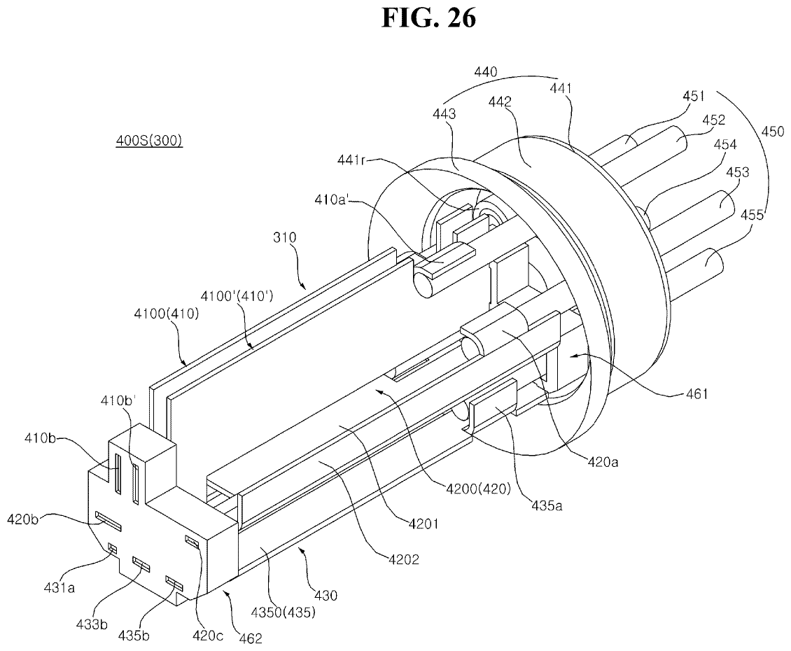

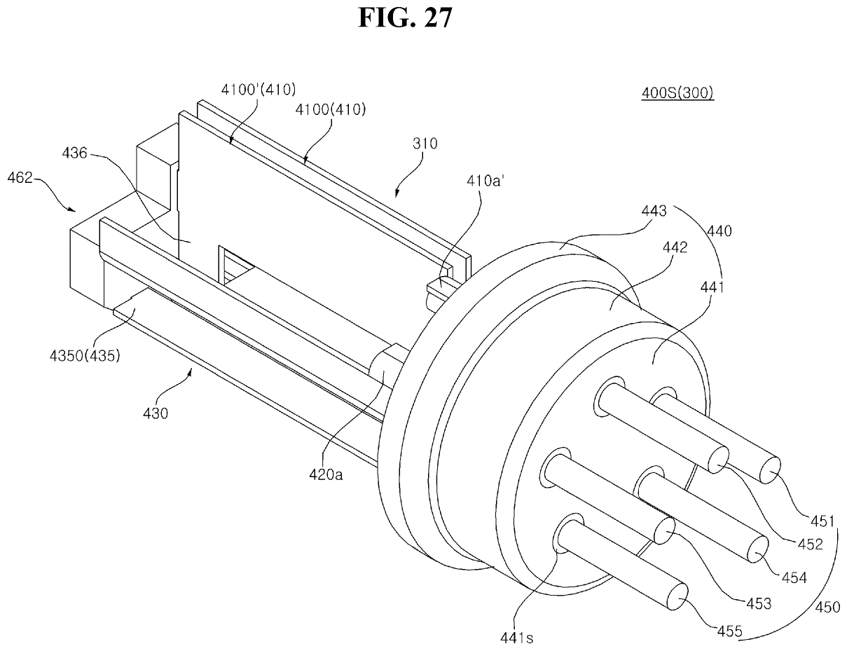

Referring to FIGS. 26 and 27, a first holder 461 may be disposed between the bottom 441 of the housing 440 and the electrode assembly 310. The first holder 461 may include an electrically insulating material such as plastic. For example, the first holder 461 may be polyphenylene sulfide (PPS). At least a portion of the plurality of pins 451, 452, 453, 454, 455 may penetrate the first holder 461. For example, the fourth and fifth pins 454, 455 may penetrate the first holder 461. Accordingly, the first holder 461 may minimize the movement of the pin assembly 450. The first holder 461 may be referred to as a pin fixing portion 461 or a first electrode fixing portion 461.

A second holder 462 may be located opposite the first holder 461 with respect to the electrode assembly 310. The second holder 462 may include an electrically insulating material such as plastic. For example, the second holder 462 may be polyphenylene sulfide (PPS). At least some of the plurality of electrodes 410, 410′, 420, 431, 433, 435 may be inserted into the second holder 462. All of the protrusion portions 410b, 410b′, 420b, 420c, 431b, 433b, 435b of the plurality of electrodes 410, 410′, 420, 431, 433, 435 may be inserted into the second holder 461. Accordingly, the second holder 462 may minimize the movement of the electrode assembly 310. The second holder 462 may be referred to as a second electrode fixing portion 462.

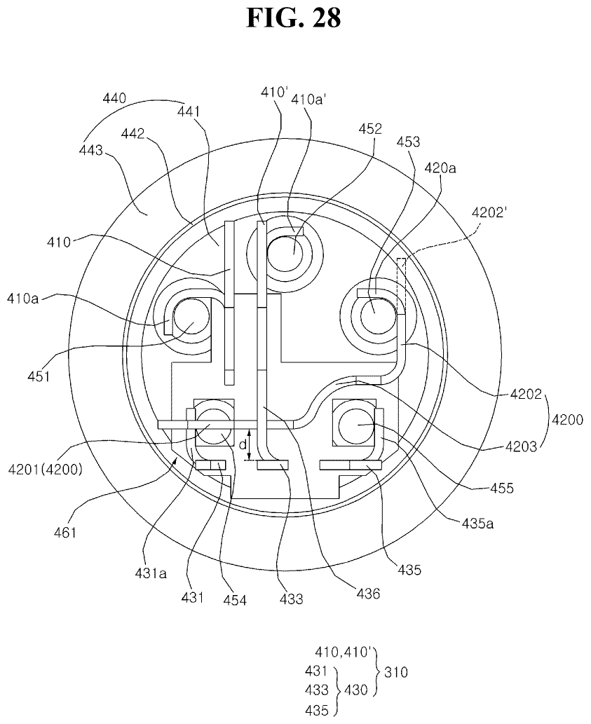

Referring again to FIG. 28, the pin assembly 450 may penetrate the bottom 441 of the housing 440, and the electrode assembly 310 may be coupled to the pin assembly 450. The electrode assembly 310 may be disposed inside the edge of the housing 440. The electrode assembly 310 may be disposed inside the circle formed by the side wall 442 of the housing 440. Accordingly, during the process of welding the housing 440 to the compressor 1 (see FIG. 22), the short circuit of the sensor module 400S may be prevented. In addition, the increase in the size of the sensor module 400S may be minimized by limiting the width (diameter) of the sensor module 400S to the width (diameter) of the housing 440.

The shield electrode 420 may include a horizontal part 4201 and a vertical part 4202. The shield electrode 420 may further include a connection part 4203.

The horizontal part 4201 may be disposed between the upper electrode assembly 410, 410′ and the lower electrode assembly 430 and may extend horizontally. The horizontal part 4201 may cover lower edges of the upper electrode assembly 410, 410′. The horizontal part 4201 may cover at least a portion of an upper surface of the lower electrode assembly 430. For example, the horizontal part 4201 may cover an upper surface of the first lower electrode 431 and an upper surface of the ground electrode 433.

The vertical part 4202 may intersect the horizontal part 4201. The vertical part 4202 may be extended in a vertical direction. The vertical part 4202 may be bent from the horizontal part 4201 to face the ground electrode 410′. The vertical part 4202 may be located opposite the upper electrode 410 with respect to the ground electrode 410′. The coupling portion 420a may be bent from the vertical part 4202. The vertical part 4202 may be extended to the boundary of a circle formed by the side wall 442 of the housing 440 (see drawing reference numeral 4202′ of FIG. 28).

The connection part 4203 may be formed between the horizontal part 4201 and the vertical part 4202. The connection part 4203 may be bent toward the center of the housing 440 from the horizontal part 4201 and the vertical part 4202. The connection part 4203 may be configured to avoid interference between the shield electrode 420 and the fifth pin 455. The connection part 4203 may be curved or angled. For example, the horizontal part 4201 may cover the upper surface of the first lower electrode 431 and the upper surface of the ground electrode 433, and the connection part 4203 may cover the upper surface of the second lower electrode 435. For example, the vertical part 4202 may be located opposite the ground electrode 410′ with respect to the third pin 453.

Accordingly, the shield electrode 420 having the vertical part 4202 may minimize the influence of the ground electrode 410′ of the upper electrode assembly 410, 410′ on the lower electrode assembly 403.

The shape and disposition of the lower electrode assembly 430 of FIG. 28 may correspond to the shape and disposition of the lower electrode assembly 431, 433, 435 described above with reference to reference numerals 1002 and 1003 of FIG. 14. In this connection, the number of pins of the pin assembly 450 may be five, and may be electrically connected to the upper electrode assembly 410, 410′, the shield electrode 420, and the lower electrode assembly 431, 433, 435 (herein, the ground electrodes 410′ and 433 may be connected to one pin). Alternatively, the pin connected to the shield electrode 420 may be omitted, in which case the number of pins of the pin assembly 450 may be four.

The lower electrode assembly 430 of the sensing module 400S of FIGS. 22 to 28 may be changed to the lower electrode assembly 431, 433, 435 of reference numeral 1302 of FIG. 17. In this connection, the number of pins of the pin assembly 450 may be five, and may be electrically connected to the upper electrode assembly 410, 410′, the shield electrode 420, and the lower electrode assembly 431, 433, 435 (herein, the ground electrodes 410′ and 433 may be connected to one pin). Alternatively, the pin connected to the shield electrode 420 may be omitted, in which case the number of pins of the pin assembly 450 may be four.

The lower electrode assembly 430 of the sensing module 400S of FIGS. 22 to 28 may be changed to the lower electrode assembly 431, 433, 435 of reference numeral 1102 of FIG. 15, the lower electrode assembly 431, 433, 435 of FIG. 16, the lower electrode assembly 431, 433, 435 of reference numeral 1602 of FIG. 20, or the lower electrode assembly 431, 433, 435 of reference numeral 1702 of FIG. 21. In this connection, the number of pins of the pin assembly 450 may be five, and may be electrically connected to the upper electrode assembly 410, 410′, the shield electrode 420, and the lower electrode assembly 431, 433, 435 (herein, the ground electrodes 410′ and 433 may be connected to one pin). Alternatively, the pin connected to the shield electrode 420 may be omitted, in which case the number of pins of the pin assembly 450 may be four.

The lower electrode assembly 430 of the sensing module 400S of FIGS. 22 to 28 may be changed to the lower electrode assembly 431, 433 of FIG. 12 or the lower electrode assembly 431, 433 of FIG. 13. In this connection, the number of pins of the pin assembly 450 may be four, and may be electrically connected to the upper electrode assembly 410, 410′, the shield electrode 420, and the lower electrode assembly 431, 433 (herein, the ground electrodes 410′ and 433 may be connected to one pin). Alternatively, the pin connected to the shield electrode 420 may be omitted, in which case the number of pins of the pin assembly 450 may be three.

The lower electrode assembly 430 of the sensing module 400S of FIGS. 22 to 28 may be changed to the lower electrode assembly 431, 435 of reference numeral 1001 of FIG. 14, the lower electrode assembly 431, 435 of reference numeral 1101 of FIG. 15, the lower electrode assembly 431, 435 of reference numeral 1301 of FIG. 17, the lower electrode assembly 431, 435 of reference numeral 1401 of FIG. 18, the lower electrode assembly 431, 435 of reference numeral 1601 of FIG. 20, or the lower electrode assembly 431, 435 of reference numeral 1701 of FIG. 21. In this connection, the number of pins of the pin assembly 450 may be five, and may be electrically connected to the upper electrode assembly 410, 410′, the shield electrode 420, and the lower electrode assembly 431, 435. Alternatively, the pins connected to the shield electrode 420 may be omitted, in which case the number of pins of the pin assembly 450 may be four.

The lower electrode assembly 430 of the sensing module 400S of FIGS. 22 to 28 may be changed to the lower electrode assembly 431 of FIG. 10 or the lower electrode assembly 431 of FIG. 11. In this connection, the number of pins of the pin assembly 450 may be four, and may be electrically connected to the upper electrode assembly 410, 410′, the shield electrode 420, and the lower electrode assembly 431. Alternatively, the pins connected to the shield electrode 420 may be omitted, in which case the number of pins of the pin assembly 450 may be three.

The lower electrode assembly 430 of the sensing module 400S of FIGS. 22 to 28 may be changed to the sub-shield electrode 425 and the lower electrode assembly 431, 435 of reference numerals 1501 and 1502 of FIG. 19. In this connection, the number of pins of the pin assembly 450 may be five, and may be electrically connected to the upper electrode assembly 410, 410′, the shield electrode 420, the lower electrode assembly 431, 435, and the sub-shield electrode 425 (herein, the sub-shield electrodes 420 and 425 may be connected to one pin). Alternatively, the pin connected to the shield electrode 420 may be omitted, in which case the number of pins of the pin assembly 450 may be four.

As described above, according to at least one embodiment of the present disclosure, the concentration of the oil present inside the compressor 1 may be accurately detected.

In addition, according to at least one embodiment of the present disclosure, the height of the surface of the oil may be accurately detected based on the concentration of the oil present inside the compressor.

In addition, according to at least one embodiment of the present disclosure, the amount of oil present inside the compressor may be accurately detected.

In addition, according to at least one embodiment of the present disclosure, a plurality of electrodes may be used to provide an optimized sensor to detect the concentration of the oil, the height of the surface of the oil, and the amount of the oil together.

In addition, various examples of the sensing module of the sensor may be provided.

Referring to FIGS. 1 to 28, the sensor according an aspect of the present disclosure may include: a cap; electrodes facing the cap and spaced apart from each other; and pins penetrating the cap and electrically connected to the electrodes, wherein the electrodes may include: an upper electrode in a shape of a vertical plate; a first ground electrode facing one surface of the upper electrode; a lower electrode located below the upper electrode; and a shield electrode disposed between the upper electrode and the lower electrode, wherein the shield electrode may cover a lower edge of the upper electrode and a lower edge of the first ground electrode.

The pins may include: a first pin connected to the upper electrode; a second pin connected to the first ground electrode; a third pin connected to the shield electrode; and a fourth pin connected to the lower electrode.

The third pin may be omitted.

The pins may be disposed radially with respect to a center of the cap.

The upper electrode, the first ground electrode, the lower electrode, and the shield electrode may be disposed inward an edge of the cap.

The shield electrode may include: a horizontal part disposed between the first ground electrode and the lower electrode; and a vertical part bent from the horizontal part to face the first ground electrode.

The shield electrode may further include a connection part recessed from a portion between the horizontal part and the vertical part, and one of the pins may be adjacent to the connection part.

Each of the upper electrode, the first ground electrode, the lower electrode, and the shield electrode may include: a coupling portion that protrudes from each of the upper electrode, the first ground electrode, the lower electrode, and the shield electrode, which is in contact with each of the pins, and which is coupled to each of the pins.

The upper electrode may include a coupling portion bent from the upper electrode to surround a pin connected to the upper electrode and welded to the pin connected to the upper electrode.

Each of the upper electrode, the first ground electrode, the lower electrode, and the shield electrode may include: one end adjacent to the cap; and the other end opposite to the one end, wherein the one end may be hung on the pin.

The sensor may further include a holder coupled to the other end of each of the upper electrode, the first ground electrode, the lower electrode, and the shield electrode.

The lower electrode may have a shape of a horizontal plate or a shape of a vertical plate, and the shield electrode may have a shape of a horizontal plate and cover an upper surface or an upper edge of the lower electrode.

The lower electrode may include: a first lower electrode; and a second lower electrode spaced apart from the first lower electrode.

The sensor may further include a second ground electrode disposed between the first lower electrode and the second lower electrode.

The sensor may further include a second ground electrode replacing the second lower electrode.

The sensor may further include: a bridge connecting the first ground electrode and the second ground electrode, wherein one of the pins may be connected to the first ground electrode or the second ground electrode.

The shield electrode may include a groove recessed from one edge of the shield electrode and through which the bridge passes.

The attached drawings are merely intended to facilitate understanding of the embodiments disclosed in this specification, and the technical ideas disclosed in this specification are not limited by the attached drawings, and should be understood to include all modifications, equivalents, or substitutes included in the spirit and technical scope of the present disclosure.

The operation method according to an embodiment of the present disclosure may be implemented as processor-readable code in a processor-readable recording medium. The processor-readable recording medium may include all types of recording devices storing data readable by a processor. Examples of processor-readable recording media include ROM, RAM, CD-ROM, magnetic tape, floppy disks, and optical data storage devices. In addition, the examples include a carrier-wave type implementation such as a transmission over the Internet. Furthermore, as the processor-readable recording medium is distributed to a computer system connected via a network, processor-readable code may be saved and executed in a distributed manner.

Further, even though preferred embodiments of the present disclosure have been illustrated and described, the present disclosure is not restricted to a certain embodiment described above, and various modifications and variations may be made by those skilled in the art without departing from the subject matter of the present disclosure as defined by the claims. Furthermore, these modified embodiments should not be understood separately from the spirit or scope of the present disclosure.

What is claimed is:

1. A sensor comprising:
a cap;
electrodes facing the cap and spaced apart from each other; and
pins penetrating the cap and electrically connected to the electrodes,
wherein the electrodes comprise:
an upper electrode in a shape of a vertical plate;
a first ground electrode facing one surface of the upper electrode;
a lower electrode located below the upper electrode; and
a shield electrode disposed between the upper electrode and the lower electrode,
wherein the shield electrode covers a lower edge of the upper electrode and a lower edge of the first ground electrode.

2. The sensor of claim 1, wherein the pins comprise:
a first pin connected to the upper electrode;
a second pin connected to the first ground electrode;
a third pin connected to the shield electrode; and
a fourth pin connected to the lower electrode.

3. The sensor of claim 2, wherein the third pin is omitted.

4. The sensor of claim 1, wherein the pins are disposed radially with respect to a center of the cap.

5. The sensor of claim 1, wherein the upper electrode, the first ground electrode, the lower electrode, and the shield electrode are disposed inward an edge of the cap.

6. The sensor of claim 1, wherein the shield electrode comprises:
a horizontal part disposed between the first ground electrode and the lower electrode; and
a vertical part bent from the horizontal part to face the first ground electrode.

7. The sensor of claim 6, wherein:
the shield electrode further comprises a connection part recessed from a portion between the horizontal part and the vertical part; and
one of the pins is adjacent to the connection part.

8. The sensor of claim 1, wherein each of the upper electrode, the first ground electrode, the lower electrode, and the shield electrode comprises a coupling portion which protrudes from each of the upper electrode, the first ground electrode, the lower electrode, and the shield electrode, which is in contact with each of the pins, and which is coupled to each of the pins.

9. The sensor of claim 1, wherein the upper electrode comprises a coupling portion bent from the upper electrode to surround a pin connected to the upper electrode and welded to the pin connected to the upper electrode.

10. The sensor of claim 1, wherein each of the upper electrode, the first ground electrode, the lower electrode, and the shield electrode comprises:
one end adjacent to the cap; and
the other end opposite to the one end,
wherein the one end is hung on the pin.

11. The sensor of claim 10, further comprising a holder coupled to the other end of each of the upper electrode, the first ground electrode, the lower electrode, and the shield electrode.

12. The sensor of claim 1, wherein:
the lower electrode has a shape of a horizontal plate or a shape of a vertical plate; and
the shield electrode has a shape of a horizontal plate and covers an upper surface or an upper edge of the lower electrode.

13. The sensor of claim 1, wherein the lower electrode comprises:
a first lower electrode; and
a second lower electrode spaced apart from the first lower electrode.

14. The sensor of claim 13, further comprising a second ground electrode disposed between the first lower electrode and the second lower electrode.

15. The sensor of claim 13, further comprising a second ground electrode replacing the second lower electrode.

16. The sensor of claim 14, further comprising a bridge connecting the first ground electrode and the second ground electrode, wherein one of the pins is connected to the first ground electrode or the second ground electrode.

17. The sensor of claim 16, wherein the shield electrode comprises a groove recessed from one edge of the shield electrode and through which the bridge passes.
